(19) United States
(12) Patent Application Publication
(10) Pub. No.: US 2026/0061172 A1
(43) Pub. Date: Mar. 5, 2026

(54) IMPEDANCE TISSUE CONFIRMATION SYSTEM

(71) Applicant: EDWARDS LIFESCIENCES CORPORATION, Irvine, CA (US)

(72) Inventors: Morgan Alex Jawitz, Mountain View, CA (US); Bezalel Haberman-Browns, Pardes Hanna (IL); Scott Louis Pool, Laguna Hills, CA (US); Sandra Ahide Alcantar Chavez, Anaheim, CA (US)

(73) Assignee: EDWARDS LIFESCIENCES CORPORATION, Irvine, CA (US)

(21) Appl. No.: 19/380,567

(22) Filed: Nov. 5, 2025

Related U.S. Application Data
(63) Continuation of application No. PCT/US2024/029606, filed on May 16, 2024.
(60) Provisional application No. 63/502,913, filed on May 17, 2023.

(51) Int. Cl. A61M 27/00

(52) U.S. Cl. CPC A61M 27/002; A61M 2205/0233; A61M 2205/3327; A61M 2205/3331; A61M 2205/50; A61M 2205/583; A61M 2210/125

(57) ABSTRACT

A device for determining tissue capture of a shunt device includes a delivery catheter and a shunt device disposed on the delivery catheter. The shunt device is configured to be inserted into a puncture in a tissue wall and includes a central flow tube extending from a first axial end to a second axial end, a first distal arm attached to the first axial end of the central flow tube, and a first proximal arm attached to the second axial end of the central flow tube. The first distal arm and the first proximal arm configured to capture the tissue wall therebetween. and the delivery catheter includes a first conductive wire and a second conductive wire. The first conductive wire is electrically connected to the shunt device and the second conductive wire is separated from the shunt device.

### CROSS-REFERENCE TO RELATED APPLICATION(S)

This application is a continuation of International Application No. PCT/US2024/029606, filed May 16, 2024, which claims the benefit of U.S. Provisional Application No. 63/502,913, filed May 17, 2023, and entitled “IMPEDANCE TISSUE CONFIRMATION SYSTEM,” the disclosure of which is hereby incorporated by reference in its entirety.

### BACKGROUND

The present disclosure relates generally to implantable devices and more specifically to confirming placement of cardiovascular shunt devices.

Shunt devices can be positioned in the heart to shunt blood between the left atrium and the right atrium to reduce pressure in the left atrium. The left atrium can experience elevated pressure due to abnormal heart conditions caused by age and/or disease. For example, shunt devices can be used to treat patients with heart failure (also known as congestive heart failure). Shunt devices can be positioned in the septal wall between the left atrium and the right atrium to shunt blood from the left atrium into the right atrium, thus reducing the pressure in the left atrium.

### SUMMARY

In one aspect, a device for determining tissue capture of a shunt device includes a delivery catheter and a shunt device disposed on the delivery catheter. The shunt device is configured to be inserted into a puncture in a tissue wall and includes a central flow tube extending from a first axial end to a second axial end, a first distal arm attached to the first axial end of the central flow tube, and a first proximal arm attached to the second axial end of the central flow tube. The first distal arm and the first proximal arm configured to capture the tissue wall therebetween. The delivery catheter includes a first conductive wire and a second conductive wire. The first conductive wire is electrically connected to the shunt device and the second conductive wire is separated from the shunt device.

In another aspect, a method of determining tissue capture of a shunt device includes deploying a first distal arm of the shunt device into a puncture in a tissue wall, moving the first proximal arm toward the first distal arm to place the first proximal arm in a deployed position relative to the first distal arm, providing an alternating current signal to a first electrode, determining an electrical impedance between the first electrode and a second electrode, and determining if tissue has been captured between the first distal arm and the first proximal arm based on the electrical impedance. The shunt device includes central flow tube with the first distal arm disposed at a first axial end of the central flow tube and the first proximal arm disposed at a second axial end of the central flow tube. The first distal arm and the first proximal arm are configured to capture tissue walls therebetween. The first distal arm is free to engage the tissue wall when deployed. The deployed position of the first proximal arm is configured to capture the tissue wall the first distal arm and the first proximal arm if the tissue wall is present. One of the first electrode and the second electrode is disposed adjacent to the first proximal arm and the other of the first electrode and the second electrode is on the first distal arm.

The present summary is provided only by way of example, and not limitation. Other aspects of the present disclosure will be appreciated in view of the entirety of the present disclosure, including the entire text, claims and accompanying figures.

### BRIEF DESCRIPTION OF THE DRAWINGS

### Anatomy of Heart H and Vasculature V

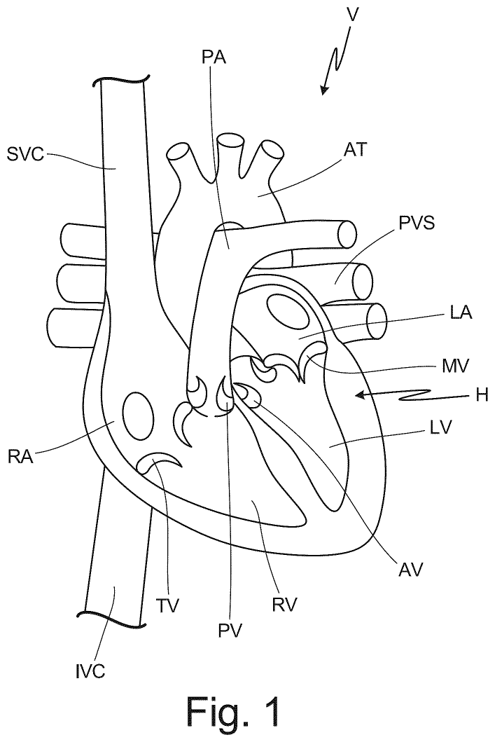

FIG. 1 is a schematic diagram of a heart and vasculature.

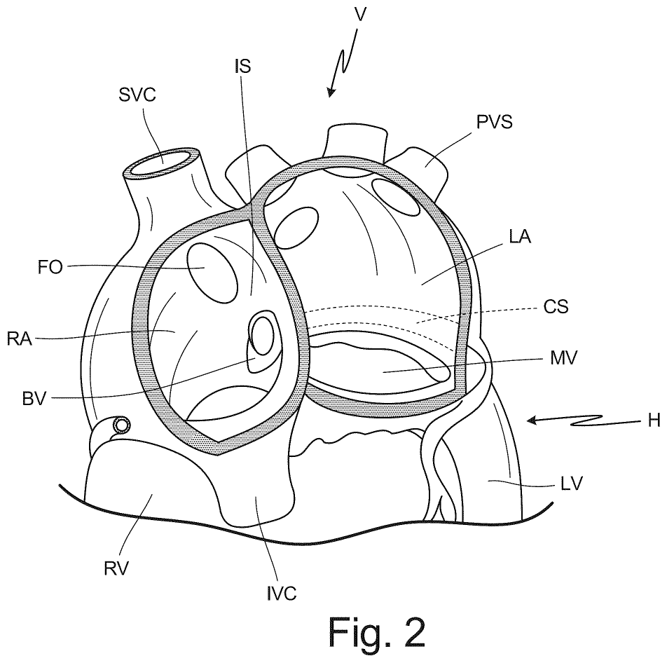

FIG. 2 is a schematic cross-sectional view of the heart.

### Shunt Devices 100 and 100′

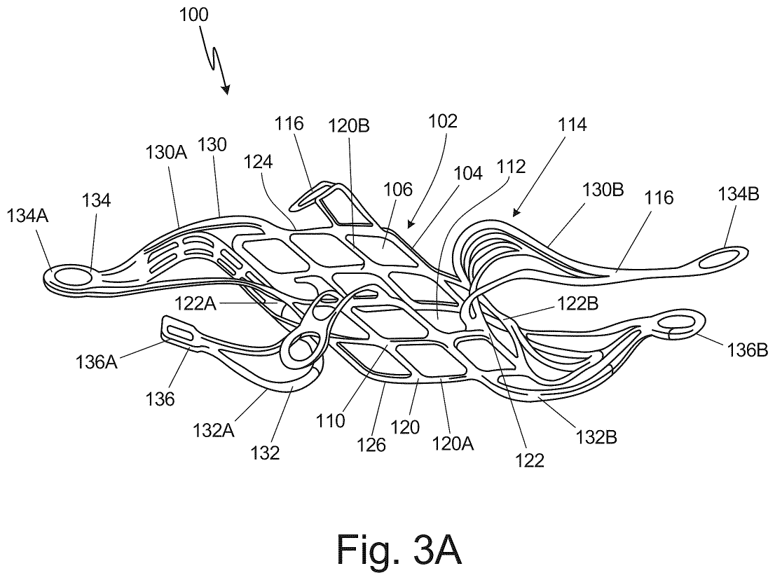

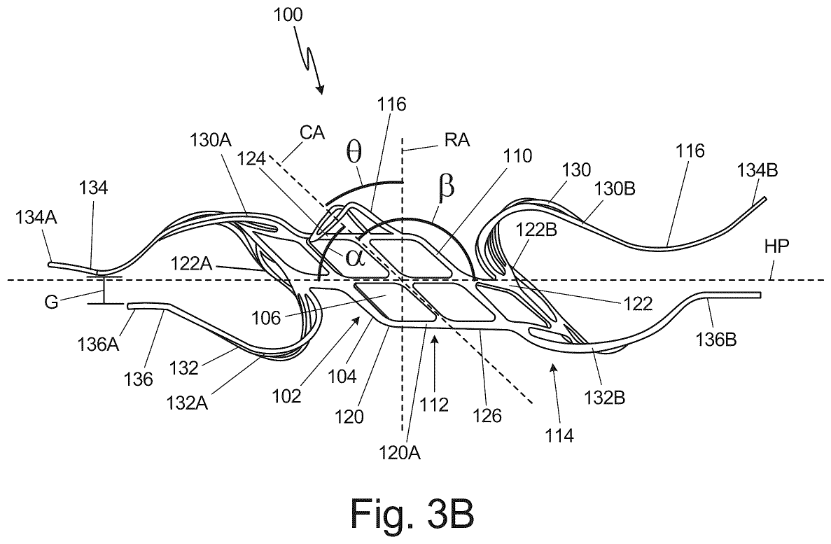

FIG. 3A is a perspective view of a shunt device.

FIG. 3B is a side view of the shunt device.

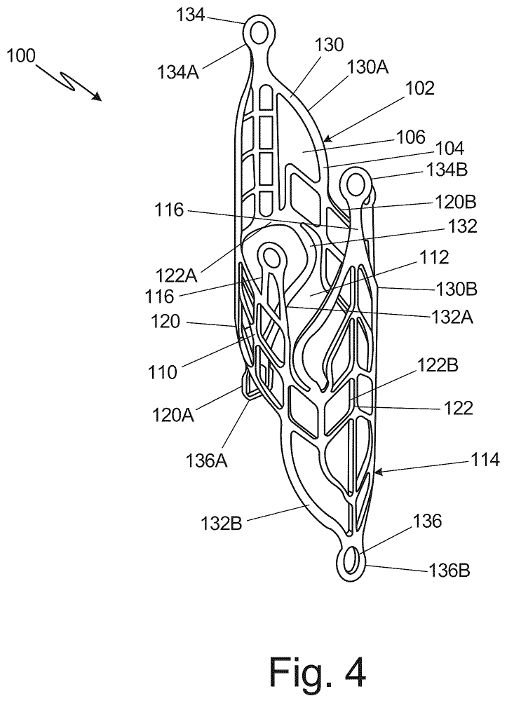

FIG. 4 is a perspective view of the shunt device in a configuration.

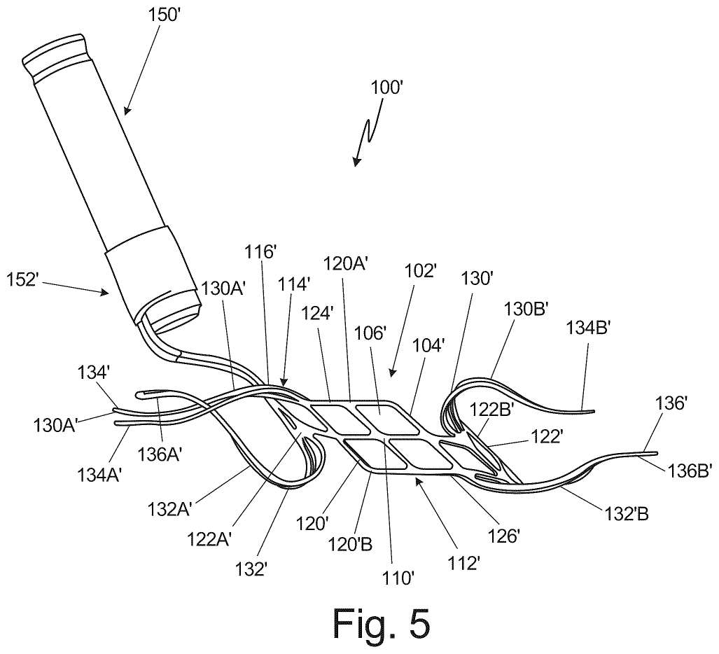

FIG. 5 is a perspective view of a shunt device including a sensor.

### Delivery Catheter 200

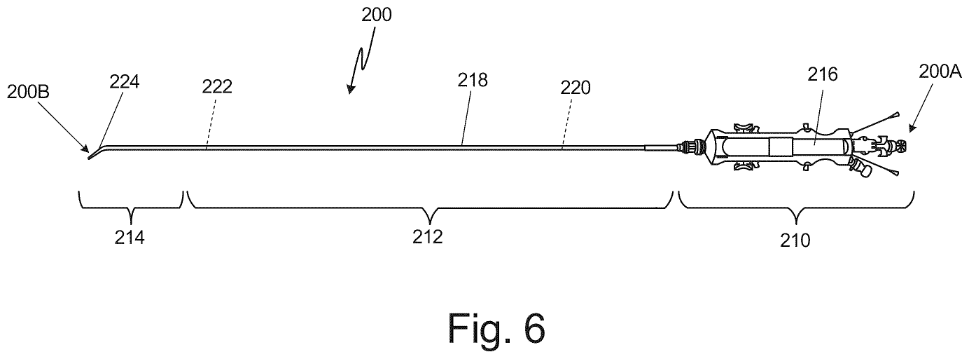

FIG. 6 is a side view of a delivery catheter.

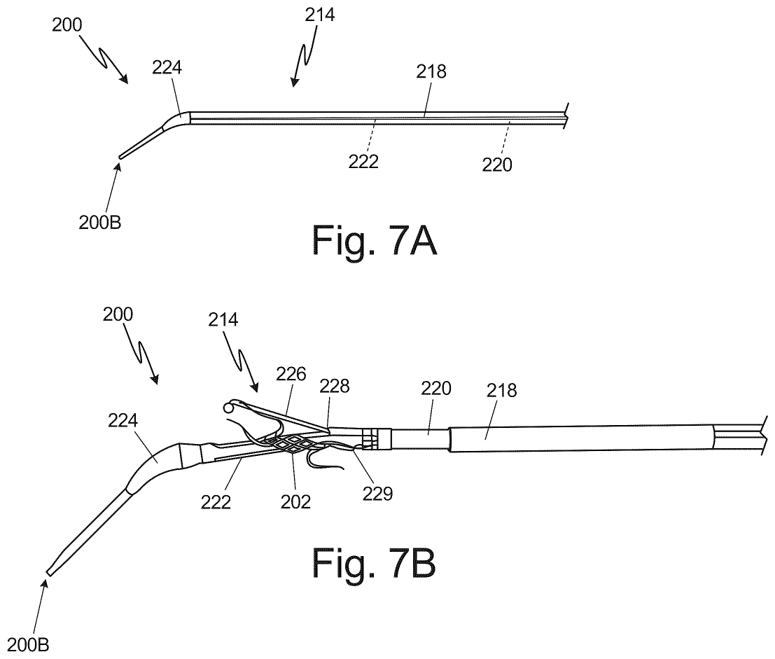

FIG. 7A is a side view of a distal portion of the delivery catheter in a sheathed state.

FIG. 7B is a side view of the distal portion of the delivery catheter in an unsheathed state.

### Delivery Method 300

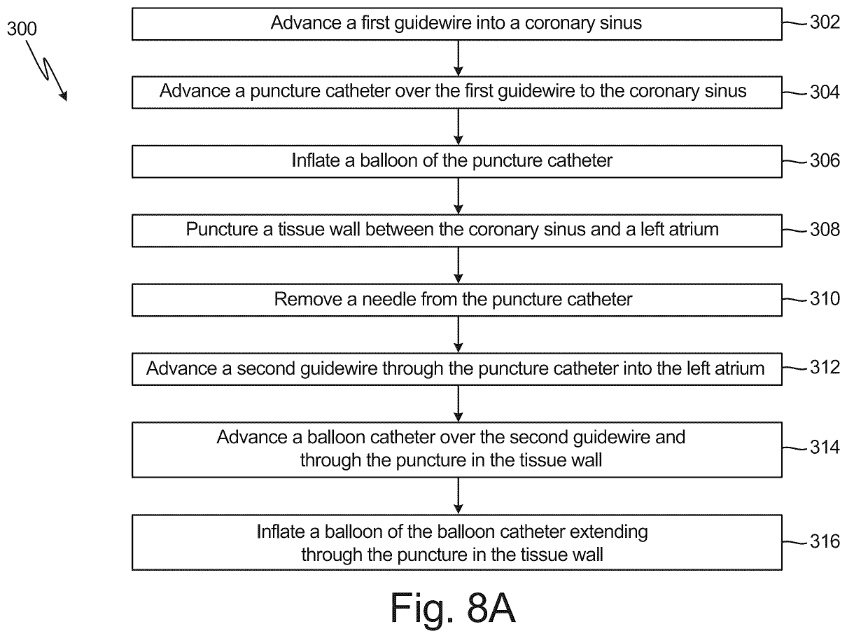

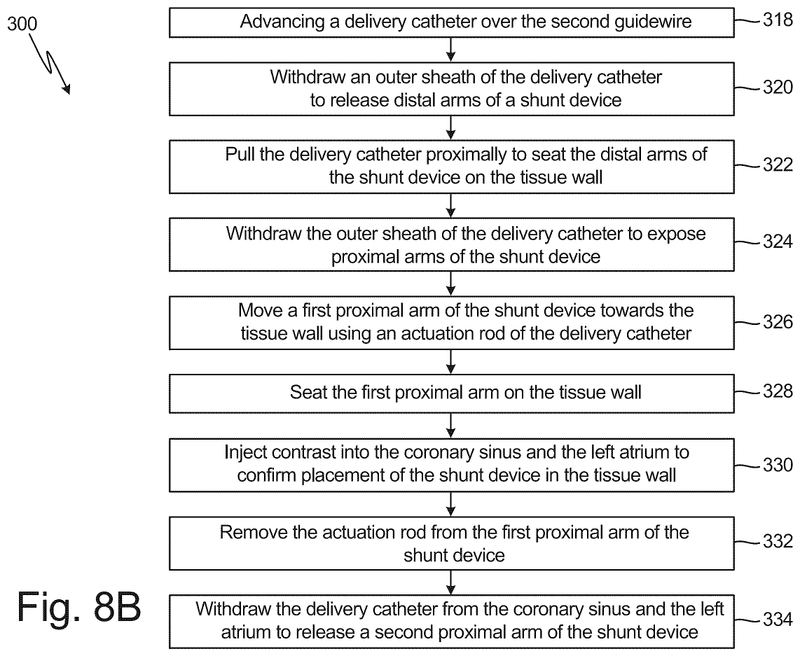

FIG. 8A is a flow chart showing steps for creating a puncture in a tissue wall between a coronary sinus and a left atrium.

FIG. 8B is a flow chart showing steps for implanting a shunt device in the tissue wall between the coronary sinus and the left atrium.

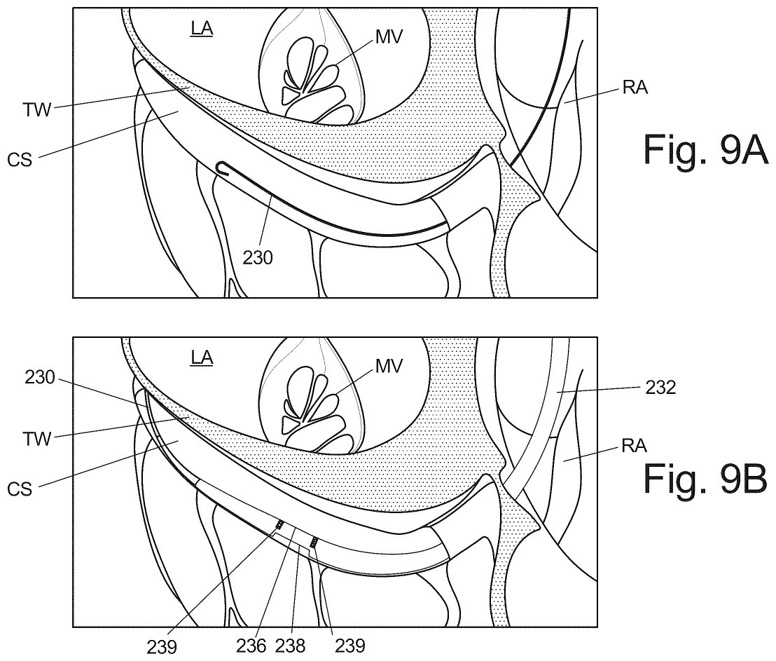

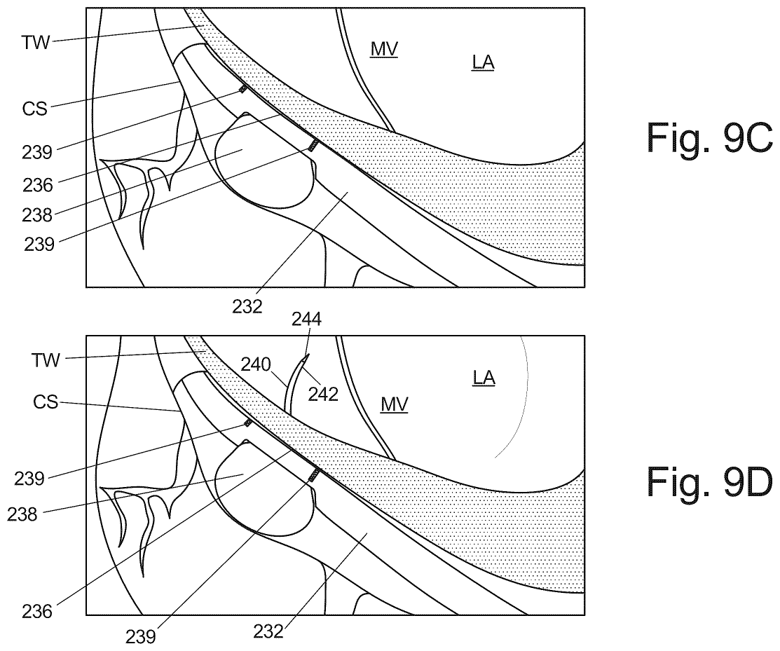

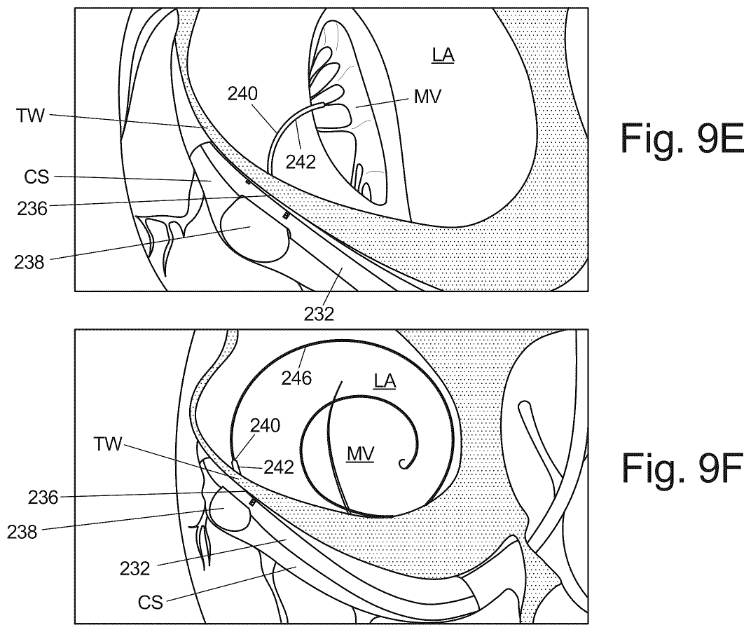

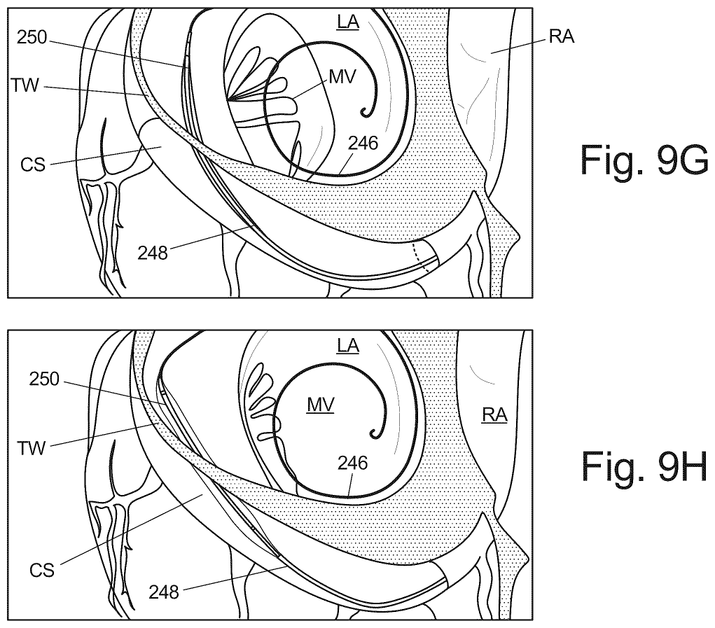

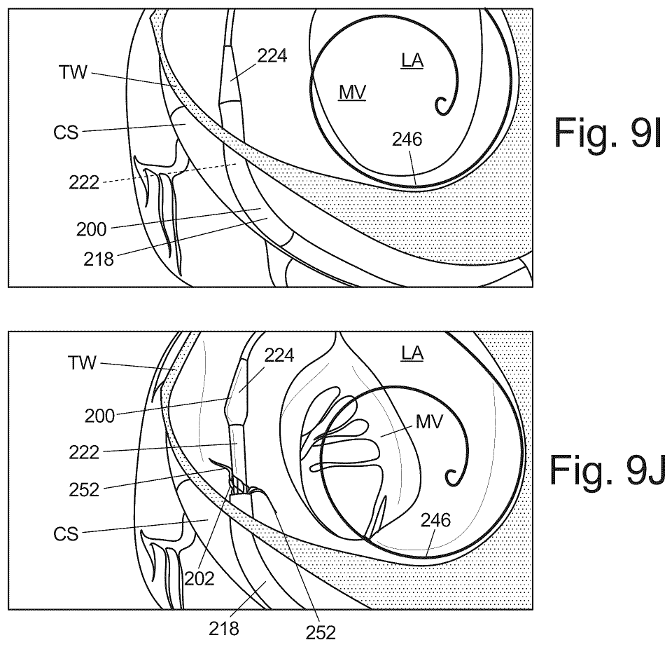

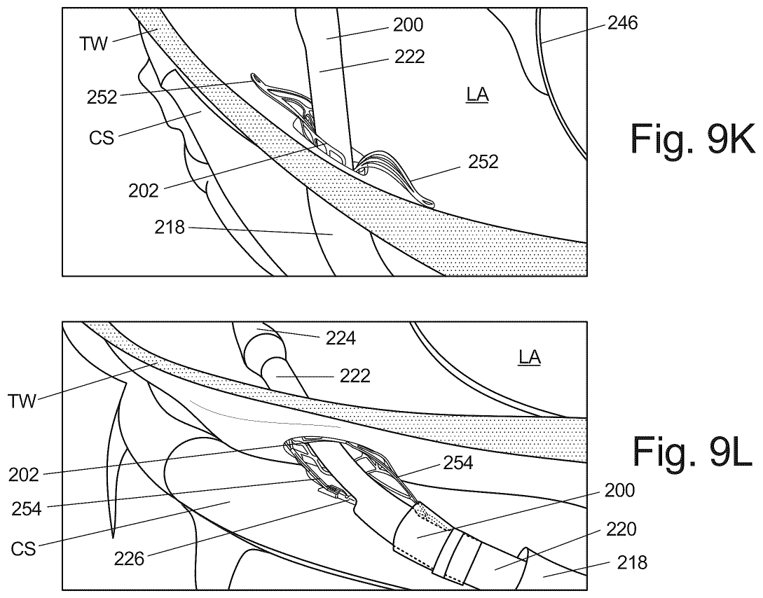

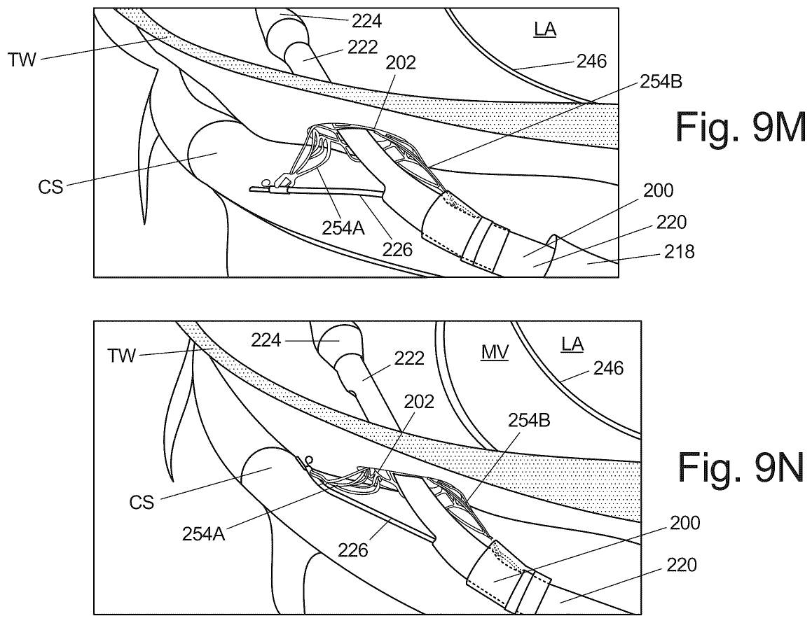

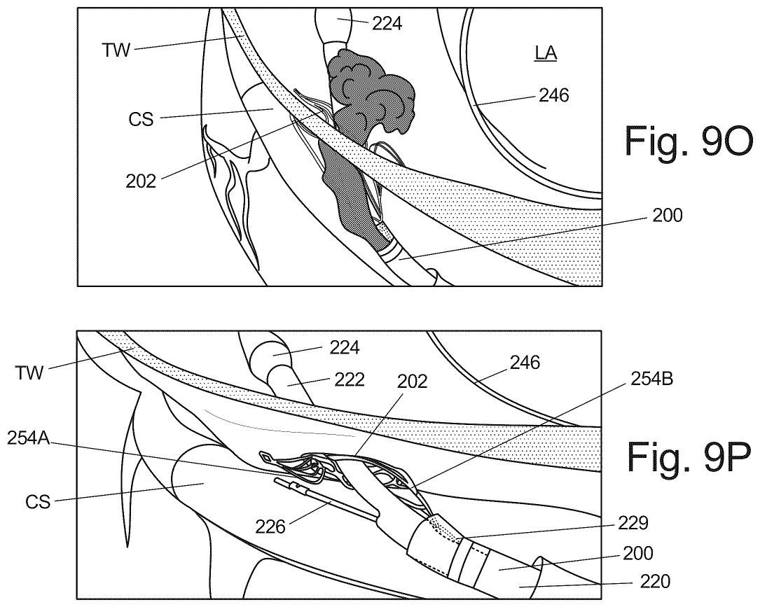

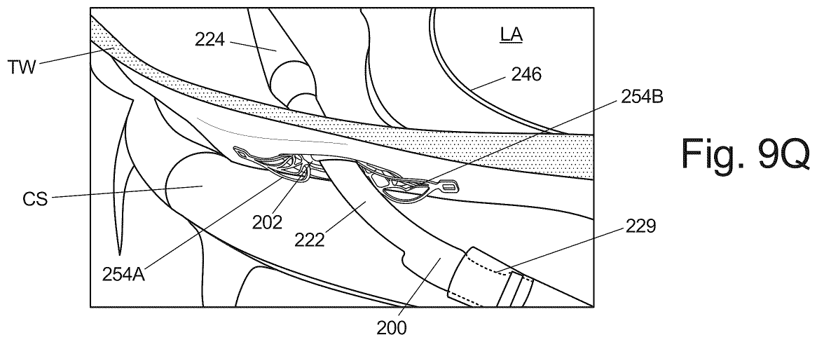

FIGS. 9A-9Q are schematic views showing the steps for implanting a shunt device in the tissue wall between the coronary sinus and the left atrium.

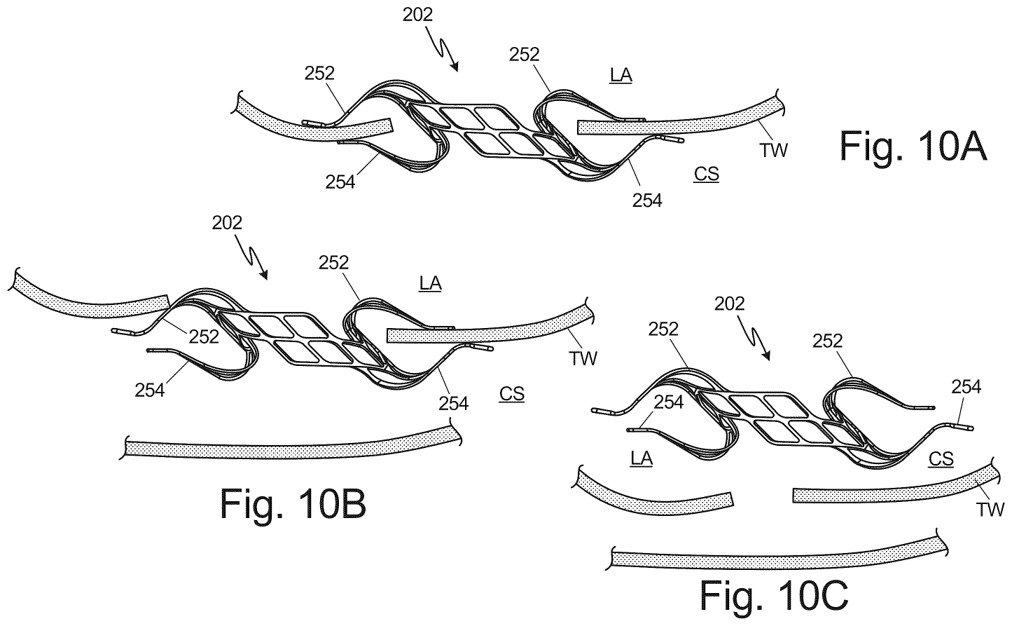

FIG. 10A is a simplified perspective view of the shunt device properly seated between the left atrium and coronary sinus.

FIG. 10B is a simplified perspective view of the shunt device improperly seated between the left atrium and coronary sinus.

FIG. 10C is a simplified perspective view of the shunt device embolized in a left atrium.

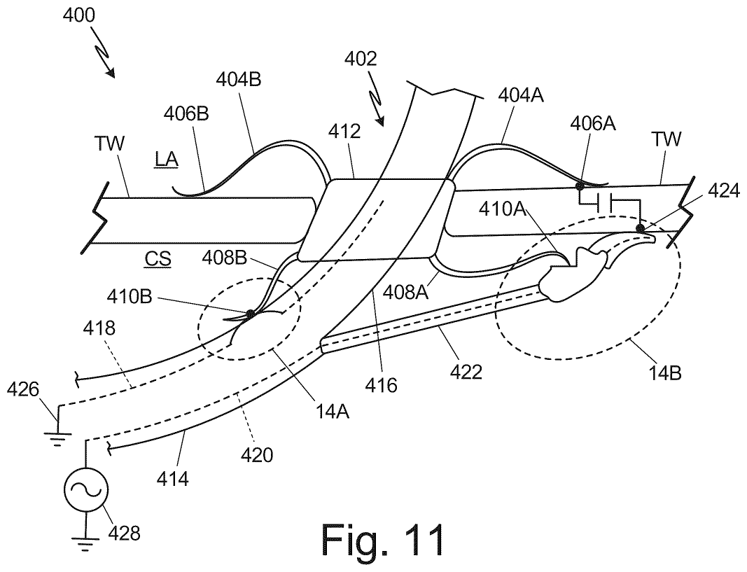

### Device and Method for Confirming Tissue Capture (FIGS. 11-15)

FIG. 11 is a simplified view of a device for determining tissue capture of a shunt device during a process of determining tissue capture, wherein tissue capture is confirmed.

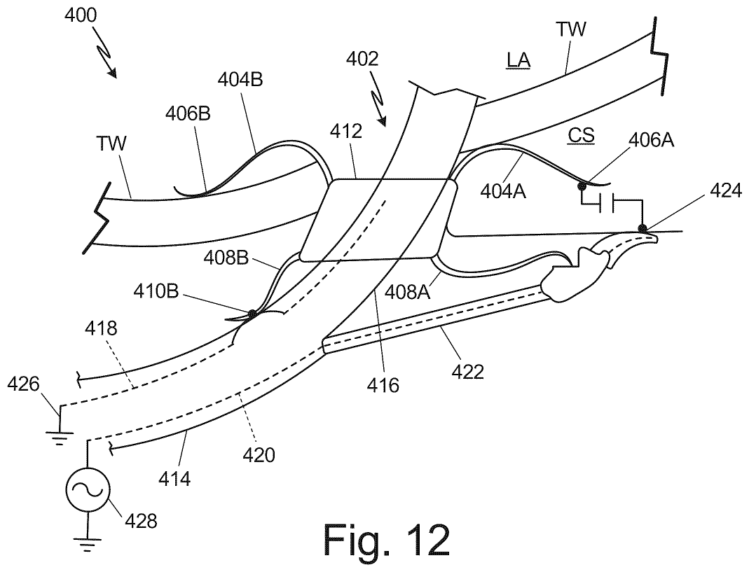

FIG. 12 is a simplified view of the device of FIG. 11 during a process of determining tissue capture, wherein tissue capture is not confirmed.

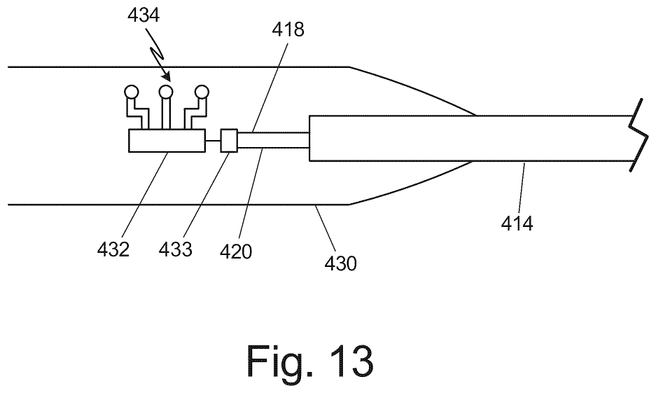

FIG. 13 is a simplified view of a handle of the device of FIG. 11.

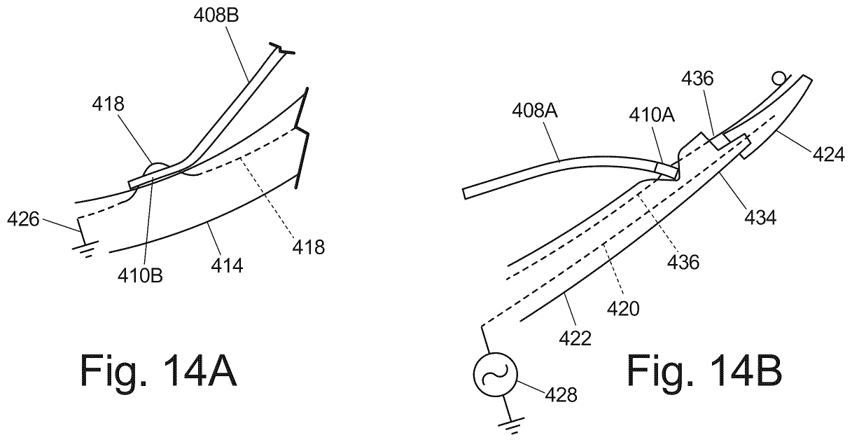

FIG. 14A is an enlarged view of portion 14A of FIG. 11.

FIG. 14B is an enlarged view of portion 14B of FIG. 11.

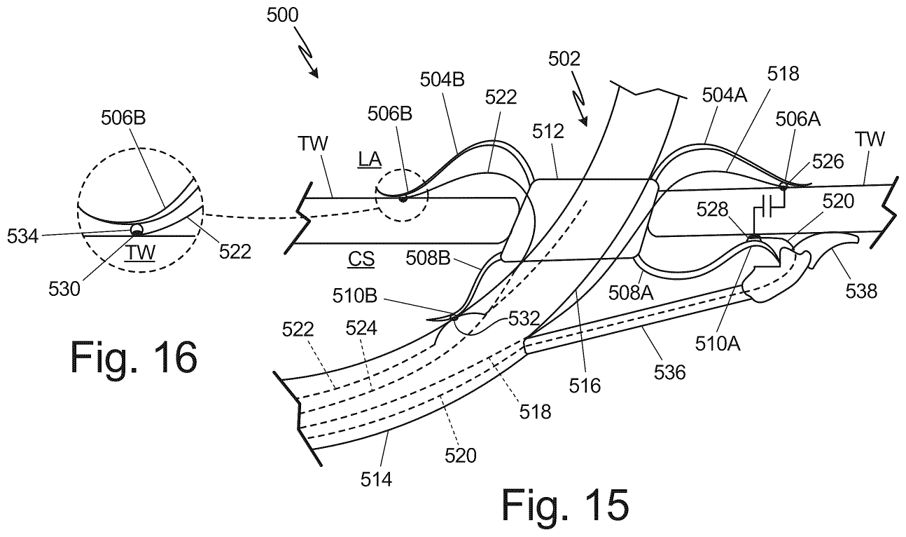

FIG. 15 is a simplified view of a device for determining tissue capture of a shunt device during deployment.

FIG. 16 is an enlarged view of a portion of FIG. 15.

While the above-identified figures set forth embodiments of the present invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale, and applications and embodiments of the present invention may include features, steps and/or components not specifically shown in the drawings.

### DETAILED DESCRIPTION

### Anatomy of Heart H and Vasculature V (FIGS. 1-2)

FIG. 1 is a schematic diagram of heart H and vasculature V. FIG. 2 is a cross-sectional view of heart H. FIGS. 1-2 will be described together. FIGS. 1-2 show heart H, vasculature V, right atrium RA, right ventricle RV, left atrium LA, left ventricle LV, superior vena cava SVC, inferior vena cava IVC, tricuspid valve TV (shown in FIG. 1), pulmonary valve PV (shown in FIG. 1), pulmonary artery PA (shown in FIG. 1), pulmonary veins PVS, mitral valve MV, aortic valve AV (shown in FIG. 1), aorta AT (shown in FIG. 1), coronary sinus CS (shown in FIG. 2), thebesian valve BV (shown in FIG. 2), inter-atrial septum IS (shown in FIG. 2), and fossa ovalis FO (shown in FIG. 2).

Heart H is a human heart that receives blood from and delivers blood to vasculature V. Heart H includes four chambers: right atrium RA, right ventricle RV, left atrium LA, and left ventricle LV.

The right side of heart H, including right atrium RA and right ventricle RV, receives deoxygenated blood from vasculature V and pumps the blood to the lungs. Blood flows into right atrium RA from superior vena cava SVC and inferior vena cava IVC. Right atrium RA pumps the blood through tricuspid valve TV into right ventricle RV. The blood is then pumped by right ventricle RV through pulmonary valve PV into pulmonary artery PA. The blood flows from pulmonary artery PA into arteries that delivery the deoxygenated blood to the lungs via the pulmonary circulatory system. The lungs can then oxygenate the blood.

The left side of heart H, including left atrium LA and left ventricle LV, receives the oxygenated blood from the lungs and pumps the blood to the body. Blood flows into left atrium LA from pulmonary veins PVS. Left atrium LA pumps the blood through mitral valve MV into left ventricle LV. The blood is then pumped by left ventricle LV through aortic valve AV into aorta AT. The blood flows from aorta AT into arteries that deliver the oxygenated blood to the body via the systemic circulatory system.

Blood is additionally received in right atrium RA from coronary sinus CS. Coronary sinus CS collects deoxygenated blood from the heart muscle and delivers it to right atrium RA. Thebesian valve BV is a semicircular fold of tissue at the opening of coronary sinus CS in right atrium RA. Coronary sinus CS is wrapped around heart H and runs in part along and beneath the floor of left atrium LA right above mitral valve MV, as shown in FIG. 2. Coronary sinus CS has an increasing diameter as it connects to right atrium RA.

Inter-atrial septum IS and fossa ovalis FS are also shown in FIG. 2. Inter-atrial septum IS is the wall that separates right atrium RA from left atrium LA. Fossa ovalis FS is a depression in inter-atrial septum IS in right atrium RA. At birth, a congenital structure called a foramen ovale is positioned in inter-atrial septum IS. The foramen ovale is an opening in inter-atrial septum IS that closes shortly after birth to form fossa ovalis FS. The foramen ovale serves as a functional shunt in utero, allowing blood to move from right atrium RA to left atrium LA to then be circulated through the body. This is necessary in utero, as the lungs are in a sack of fluid and do not oxygenate the blood. Rather, oxygenated blood is received from the mother. The oxygenated blood from the mother flows from the placenta into inferior vena cava IVC through the umbilical vein and the ductus venosus. The oxygenated blood moves through inferior vena cava IVC to right atrium RA. The opening of inferior vena cava IVC in right atrium RA is positioned to direct the oxygenated blood through right atrium RA and the foramen ovale into left atrium LA. Left atrium LA can then pump the oxygenated blood into left ventricle LV, which pumps the oxygenated blood to aorta AT and the systemic circulatory system. This allows the pulmonary circulatory system to be bypassed in utero. Upon birth, respiration expands the lungs, blood begins to circulate through the lungs to be oxygenated, and the foramen ovale closes to form fossa ovalis FS.

Shunt devices can be positioned in heart H to shunt blood between left atrium LA and right atrium RA. Left atrium LA can experience elevated pressure due to abnormal heart conditions. It has been hypothesized that patients with elevated pressure in left atrium LA may benefit from a reduction of pressure in left atrium LA. Shunt devices can be used in these patients to shunt blood from left atrium LA to right atrium RA to reduce the pressure of blood in left atrium LA, which reduces the systolic preload on left ventricle LV. Reducing pressure in left atrium LA further relieves back-pressure on the pulmonary circulation to reduce the risk of pulmonary edema.

For example, shunt devices can be used to treat patients with heart failure (also known as congestive heart failure). The hearts of patients with heart failure do not pump blood as well as they should. Heart failure can affect the right side and/or the left side of the heart. Diastolic heart failure (also known as heart failure with preserved ejection fraction) refers to heart failure occurring when the left ventricle is stiff (having less compliance), which makes it hard to relax appropriately and fill with blood. This leads to increased end-diastolic pressure, which causes an elevation of pressure in left atrium LA. There are very few, if any, effective treatments available for diastolic heart failure. Other examples of abnormal heart conditions that cause elevated pressure in left atrium LA are systolic dysfunction of the left ventricle and valve disease.

Septal shunt devices (also called inter-atrial shunt devices) are positioned in inter-atrial septum IS to shunt blood directly from left atrium LA to right atrium RA. Typically, septal shunt devices are positioned in fossa ovalis FS, as fossa ovalis FS is a thinner area of tissue in inter-atrial septum IS where the two atria share a common wall. If the pressure in right atrium RA exceeds the pressure in left atrium LA, septal shunt devices can allow blood to flow from right atrium RA to left atrium LA. This causes a risk of paradoxical stroke (also known as paradoxical embolism), as emboli can move from right atrium RA to left atrium LA and then into aorta AT and the systemic circulation.

Shunt devices can also be left atrium to coronary sinus shunt devices that are positioned in a tissue wall between left atrium LA and coronary sinus CS where the two structures are in close approximation. Left atrium to coronary sinus shunt devices move blood from left atrium LA into coronary sinus CS, which then delivers the blood to right atrium RA via thebesian valve BV, the natural orifice of coronary sinus CS. Coronary sinus CS acts as an additional compliance chamber when using a left atrium to coronary sinus shunt device. Left atrium to coronary sinus shunt devices further provide increased protections against paradoxical strokes, as the blood would have to flow retrograde from right atrium RA through coronary sinus CS before entering left atrium LA. Further, left atrium to coronary sinus shunt devices also provide protection against significant right atrium RA to left atrium LA shunting, as again the blood would have to flow retrograde from right atrium RA through coronary sinus CS before entering left atrium LA.

### Shunt Devices 100 and 100′ (FIGS. 3A-5)

FIG. 3A is a perspective view of shunt device 100. FIG. 3B is a side view of shunt device 100. FIG. 4 is a perspective view of shunt device 100 in a collapsed configuration. FIGS. 3A, 3B, and 4 will be described together. Shunt device 100 includes body 102, which is formed of struts 104 and openings 106. Body 102 includes central flow tube 110, flow path 112, and arms 114. Shunt device 100 also includes tissue capture features 116. Central flow tube 110 has side portions 120 (including side portion 120A and side portion 120B), end portions 122 (including end portion 122A and end portion 122B), first axial end 124, and second axial end 126. Arms 114 include distal arms 130 (including distal arm 130A and distal arm 130B) and proximal arms 132 (including proximal arm 132A and proximal arm 132B). Distal arms 130 have terminal ends 134 (including terminal end 134A and terminal end 134B). Proximal arms 132 have terminal ends 136 (including terminal end 136A and terminal end 136B). FIG. 3B further shows gap G, horizontal reference plane HP, perpendicular reference axis RA, central axis CA, tilt angle θ, first angle α, and second angle β.

Shunt device 100 is a cardiovascular shunt. Shunt device 100 is shown in an expanded configuration in FIGS. 3A-3B. Shunt device 100 is formed of a super-elastic material that is capable of being compressed into a catheter for delivery into the body that can then retain its relaxed, or expanded, shape when it is released from the catheter. For example, shunt device 100 can be formed of a shape-memory material, such as nitinol (a nickel titanium alloy). Shunt device 100 is shown in a compressed configuration in FIG. 4. Upon delivery into the body, shunt device 100 will expand back to its relaxed, or expanded, shape. Shunt device 100 can be sterilized before being delivered into the body. Shunt device 100 has body 102 that is formed of interconnected struts 104. Openings 106 in body 102 are defined by struts 104. Body 102 of shunt device 100 is formed of struts 104 to increase the flexibility of shunt device 100 to enable it to be compressed and expanded.

Body 102 includes central flow tube 110 that forms a center portion of shunt device 100. Central flow tube 110 is tubular in cross-section but is formed of struts 104 and openings 106. Central flow tube 110 can be positioned in a puncture or opening in a tissue wall and hold the puncture open. Flow path 112 is an opening extending through central flow tube 110. Flow path 112 is the path through which blood flows through shunt device 100 when shunt device 100 is implanted in the body. Arms 114 extend from central flow tube 110. Arms 114 extend outward from central flow tube 110 when shunt device 100 is in an expanded configuration. Arms 114 hold shunt device 100 in position in the tissue wall when shunt device 100 is implanted in the body.

When shunt device 100 is implanted in the tissue wall between the left atrium and the coronary sinus of the heart, central flow tube 110 holds the puncture open so blood can flow from the left atrium to the coronary sinus through flow path 112. Struts 104 of central flow tube 110 form a lattice or cage of sorts that is sufficient to hold the puncture in the tissue wall open around central flow tube 110. Central flow tube 110 extends from first axial end 124 to second axial end 126. Central flow tube 110 is designed to have an axial length, as measured from first axial end 124 to second axial end 126, that approximates the thickness of the tissue wall between the left atrium and the coronary sinus. When shunt device 100 is implanted in the tissue wall between the left atrium and the coronary sinus, first axial end 124 can be facing the left atrium (i.e., a left atrial side of shunt device 100) and second axial end 126 can be facing the coronary sinus (i.e., a coronary sinus side of shunt device 100). In other examples, the orientation of first axial end 124 and second axial end 126 can be reversed.

Central flow tube 110 has side portions 120 and end portions 122. Side portion 120A and side portion 120B form opposing sides of central flow tube 110. End portion 122A and end portion 122B form opposing ends of central flow tube 110. End portion 122A and end portion 122B each extend between and connect to side portion 120A and side portion 120B to form a generally circular or oval opening that defines flow path 112. Side portions 120 and end portions 122 form a tubular lattice for central flow tube 110. Struts 104 of central flow tube 110 define openings 106 in central flow tube 110. In some examples, openings 106 can be generally parallelogram-shaped. In other examples, openings 106 can be any regular or irregular shape as desired. For example, struts 104 of side portions 120 can form an array of parallelogram-shaped openings 106 in side portions 120. Struts 104 of end portions 122 can form openings 106 in end portions 122. Struts 104 of arms 114 can form openings 106 in arms 114.

As shown in FIG. 3B, central flow tube 110 is angled with respect to horizontal reference plane HP extending through shunt device 100. Horizontal reference plane HP lies generally in the plane of the tissue wall immediately adjacent to shunt device 100 when shunt device 100 is implanted in the tissue wall. End portions 122 are similarly angled with respect to horizontal reference plane HP. Perpendicular reference axis RA, as shown in FIG. 3B, is perpendicular to horizontal reference plane HP. As shown in FIG. 3B, central axis CA is an axis through the center of central flow tube 110 and flow path 112. Central axis CA extends through central flow tube 110 at tilt angle θ with respect to perpendicular reference axis RA. Accordingly, central axis CA defines the angle or tilt of central flow tube 110 with respect to perpendicular reference axis RA (and horizontal reference plane HP). End portions 122 of central flow tube 110 extend parallel to central axis CA.

Arms 114 of shunt device 100 include two distal arms 130 and two proximal arms 132. In some examples, individual ones of distal arms 130 and/or proximal arms 132 can be formed of multiple split arm portions. Arms 114 extend outward from end portions 122 of central flow tube 110 when shunt device 100 is in an expanded configuration. Distal arm 130A is connected to and extends away from end portion 122A, and distal arm 130B is connected to and extends away from end portion 122B. Proximal arm 132A is connected to and extends away from end portion 122A, and proximal arm 132B is connected to and extends away from end portion 122B. When shunt device 100 is implanted in the tissue wall between the left atrium and the coronary sinus, distal arms 130 will be positioned in the left atrium and proximal arms 132 will be positioned in the coronary sinus. Distal arms 130 each have terminal ends 134. Specifically, distal arm 130A has terminal end 134A, and distal arm 130B has terminal end 134B. Proximal arms 132 each have terminal ends 136. Specifically, proximal arm 132A has terminal end 136A, and proximal arm 132B has terminal end 136B.

Distal arms 130 and proximal arms 132 curl outward from end walls 122. As shown in FIG. 3B, each of distal arms 130 and proximal arms 132 has a proximal portion adjacent to central flow tube 110 that forms a shallow curve or arc in a direction away from end walls 122 of central flow tube 110. Each of distal arms 130 and proximal arms 132 flattens out towards respective terminal ends 134 and 136 such that a portion of each of distal arms 130 and proximal arms 132 at or adjacent to the respective terminal end 134 or 136 is generally parallel to horizontal reference plane HP. Accordingly, an axis drawn through terminal end 134A and an axis drawn through terminal end 136B, which are approximated in FIG. 3B as axes in the plane of horizontal reference plane HP for simplicity, can each form first angle α with central axis CA through central flow tube 110. Similarly, an axis drawn through terminal end 134B, and an axis drawn through terminal end 136A, which are approximated in FIG. 3B as axes in the plane of horizontal reference plane HP for simplicity, can each form second angle β with central axis CA through central flow tube 110. Alternatively, distal arms 130 and proximal arms 132 do not flatten out and become parallel to horizontal reference plane HP but instead approach horizontal reference plane HP at an angle and/or have respective terminal ends 134 and 136 that angle away from horizontal reference plane HP. In such examples, first angle α and second angle β are approximations of the central angle for the arcs from end walls 122 to the tissue wall that each respective arm encompasses when shunt device 100 is implanted in the tissue wall. Put more simply, first angle α is the angle between central axis CA and horizontal reference plane HP, and second angle β is the supplementary angle to first angle α. In some examples, first angle α can be less than ninety degrees (<90°) and second angle β can be greater than ninety degrees (>90°). In other examples, first angle α and second angle β can be any suitable combination of angles that add to one hundred eighty degrees (1800). The difference between first angle α and second angle β (and the corresponding curvature of ones of distal arms 130 and proximal arms 132) accommodates for the tilt of central flow tube 110.

As shown in FIG. 3B, distal arm 130A and distal arm 130B extend outwards from central flow tube 110 in opposite directions parallel to horizontal reference plane HP. Distal arm 130A and distal arm 130B can be aligned with each other (i.e., oriented at 180° to each other across central flow tube 110). In some examples, distal arm 130A has a longer length than distal arm 130B. In other examples, distal arm 130A has a shorter length than distal arm 130B. In yet other examples, distal arms 130 can have similar lengths. Proximal arm 132A and proximal arm 132B extend outwards from central flow tube 110 in opposite directions parallel to horizontal reference plane HP. Proximal arm 132A and proximal arm 132B can be aligned with each other (i.e., oriented at 180° to each other across central flow tube 110). In some examples, proximal arm 132A has a shorter length than proximal arm 132B. In other examples, proximal arm 132A has a longer length than proximal arm 132B. In yet other examples, proximal arms 132 can have similar lengths. In some examples, distal arm 130A has generally the same length and shape as proximal arm 132B, and distal arm 130B has generally the same length and shape as proximal arm 132A. In other examples, each of distal arms 130 and proximal arms 132 can have different lengths and shapes, though the overall shape of each arm is similar. As such, shunt device 100 has some degree of inverse symmetry across horizontal reference plane HP, as shown in FIG. 3B.

Shunt device 100 is generally elongated longitudinally but is relatively narrow laterally. Stated another way, distal arms 130 and proximal arms 132 are not annular or circular, but rather extend outward generally in only one plane. As shown in FIG. 3B, shunt device 100 has a generally H-shape when viewing a side of shunt device 100. The elongated shape of shunt device 100 means that when compressed it elongates along a line, as shown in FIG. 4, so as to better fit within a catheter.

Terminal ends 134 of distal arms 130 and terminal ends 136 of proximal arms 132 converge towards one another. Distal arms 130 and proximal arms 132 form two pairs of arms. That is, each of distal arms 130 forms a clamping pair with a corresponding one of proximal arms 132. Distal arm 130A and proximal arm 132A form a first pair of arms extending outward from a first side of central flow tube 110, and terminal end 134A of distal arm 130A converges towards terminal end 136A of proximal arm 132A. Distal arm 130B and proximal arm 132B form a second pair of arms extending outward from a second side of central flow tube 110, and terminal end 134B of distal arm 130B converges towards terminal end 136B of proximal arm 132B. Gap G between terminal ends 134 and terminal ends 136 is sized to be slightly smaller than an approximate thickness of the tissue wall between the left atrium and the coronary sinus, or another tissue wall of interest. This allows distal arms 130 and proximal arms 132 to flex outwards and grip the tissue wall when implanted to help hold shunt device 100 in place against the tissue wall. Thus, a distance corresponding to gap G, as measured once shunt device 100 is implanted, may be slightly different between different clamping pairs of distal arms 130 and proximal arms 132 depending on anatomical variations along the particular tissue wall. Terminal ends 134 of distal arms 130 and terminal ends 136 of proximal arms 132 can also have openings or indentations that are configured to engage a delivery tool to facilitate implantation of shunt device 100, for example actuating rods of a delivery tool. Additionally, terminal ends 134 of distal arms 130 and terminal ends of proximal arms 132 can include locations for radiopaque markers to permit visualization of the positioning of shunt device 100.

When implanted in the tissue wall, distal arms 130 and proximal arms 132 are designed such that the projection of distal arms 130 and proximal arms 132 into the left atrium and the coronary sinus, respectively, is minimized. This minimizes the disruption of the natural flow patterns in the left atrium and the coronary sinus. Shunt device 100 can also be designed so that the profile of proximal arms 132 projecting into the coronary sinus is lower than the profile of distal arms 130 projecting into the left atrium to minimize disruption of the natural blood flow through the coronary sinus and to reduce the potential for proximal arms 132 to block the narrower passage of the coronary sinus.

Tissue capture features 116 can take several different forms. For example, tissue capture features 116 connected to central flow tube 110 at first axial end 124 and/or second axial end 126 can be tabs that extend outward from side portions 120. Tissue capture features 116 connected to arms 114 can be deflectable projections that extend between respective ones of arms 114 and the tissue wall to be compressed back toward the respective arm 114 when shunt device 100 is implanted in the tissue wall. Tissue capture features 116 connected to end portions 122 of central flow tube 110 can be secondary arms associated with one of arms 114. Tissue capture features 116 that are a part of arms 114 themselves can be, e.g., a lengthened portion of one of arms 114, separate split arm portions of one of arms 114, and/or interlacing arms 114. Any one or more of tissue capture features 116 can be incorporated alone or in combination on shunt device 100 to aid in anchoring shunt device 100 to the tissue wall and to prevent displacement of shunt device 100.

FIG. 5 is a perspective view of shunt device 100′ including sensor 150′. Shunt device 100′ includes body 102′, which is formed of struts 104′ and openings 106′. Body 102′ includes central flow tube 110′, flow path 112′, arms 114′. Shunt device 100′ also includes and tissue capture features 116′. Central flow tube 110′ has side portions 120′ (including side portion 120A′ and side portion 120B′), end portions 122′ (including end portion 122A′ and end portion 122B′), first axial end 124′, and second axial end 126′. Arms 114′ include distal arms 130′ (including distal arm 130A′ and distal arm 130B′) and proximal arms 132′ (including proximal arm 132A′ and proximal arm 132B′). Distal arms 130′ have terminal ends 134′ (including terminal end 134A′ and terminal end 134B′). Proximal arms 132′ have terminal ends 136′ (including terminal end 136A′ and terminal end 136B′). Shunt device 100′ further includes sensor 150′ and sensor attachment portion 152′.

Shunt device 100′ includes a similar structure and design to shunt device 100 described above, except shunt device 100′ additionally includes sensor 150′ connected to sensor attachment portion 152′.

As shown in FIG. 5, sensor 150′ can be attached to shunt device 100′ so that sensor 150′ is positioned in the left atrium when shunt device 100′ is implanted in the tissue wall between the left atrium and the coronary sinus of the heart. Accordingly, sensor 150′ can be attached to one of distal arms 130′. Alternatively, sensor 150′ can be attached to shunt device 100′ so that sensor 150′ is positioned in the coronary sinus when shunt device 100′ is implanted in the tissue wall. In such examples, sensor 150′ can be attached to one of proximal arms 132′. In further examples, an additional sensor can be included on shunt device 100′ to position sensors in both the left atrium and the coronary sinus.

Sensor 150′ is attached to shunt device 100′ at sensor attachment portion 152′. Sensor 150′ can be connected to sensor attachment portion 152′ using any suitable attachment mechanism. For example, sensor 150′ and sensor attachment portion 152′ can include complimentary mating features. Sensor attachment portion 152′ can be an extension of one of arms 114′ of shunt device 100′. In some examples, sensor attachment portion 152′ is an extension of distal arm 130A′. In other examples, sensor attachment portion 152′ is an extension of distal arm 130B′ or one of proximal arms 132′. Alternatively, as shown in FIG. 5, sensor attachment portion 152′ can be a separate split arm portion of one of arms 114′. Sensor attachment portion 152′ can be angled away from a horizontal reference plane (not shown) that is in the plane of the tissue wall adjacent to shunt device 100′ when shunt device 100′ is implanted in the tissue wall. That is, sensor attachment portion 152′ can be angled away from the tissue wall.

Sensor 150′ can be a pressure sensor to sense a pressure in the left atrium. In other examples, sensor 150′ can be any sensor to measure a parameter in the left atrium. In yet other examples, sensor 150′ can be any sensor to measure a parameter in the coronary sinus. Sensor 150′ can include a transducer, control circuitry, and an antenna in one example. The transducer, for example a pressure transducer, is configured to sense a signal from the left atrium. The transducer can communicate the signal to the control circuitry. The control circuitry can process the signal from the transducer or communicate the signal from the transducer to a remote device outside of the body using the antenna. Sensor 150′ can include alternate or additional components in other examples. Further, the components of sensor 150′ can be held in a sensor housing that is hermetically sealed.

### Delivery Catheter 200 (FIGS. 6-7B)

FIG. 6 is a side view of delivery catheter 200. FIG. 7A is a side view of distal portion 214 of delivery catheter 200 in a sheathed state. FIG. 7B is a side view of distal portion 214 of delivery catheter 200 in an unsheathed state. FIGS. 6, 7A, and 7B will be discussed together. FIGS. 6-7B show delivery catheter 200. FIG. 7B shows shunt device 202. Delivery catheter 200 includes proximal end 200A, distal end 200B, proximal portion 210, intermediate portion 212, distal portion 214, handle 216, outer sheath 218, inner sheath 220, bridge 222, nosecone 224, actuation rod 226, side opening 228, and notch 229.

Delivery catheter 200 is one example of a delivery catheter that can be used to implant a shunt device into a patient. Delivery catheter 200 as shown in FIGS. 6-7B is used to implant shunt device 202 (shown in FIG. 7B). Delivery catheter 200 can take other forms in alternate examples. Shunt device 202 can have the structure and design of any suitable shunt device, for example shunt device 100 or 100′ as shown in FIGS. 3A-5. Delivery catheter 200 is shown as being configured to implant shunt device 202 without a sensor in the example shown in FIGS. 6-7B. In alternate examples, delivery catheter 200 can be used to implant a shunt device with a sensor, including any needed modifications to accommodate the sensor.

Delivery catheter 200 includes proximal portion 210 adjacent proximal end 200A of delivery catheter 200, intermediate portion 212 extending from proximal portion 210, and distal portion 214 extending from intermediate portion 212 to distal end 200B of delivery catheter 200. Proximal portion 210 includes handle 216, which can be grasped by a physician to control movement of delivery catheter 200. Handle 216 includes a number of ports through which guide wires, tubes, fluids, or other components or elements may be passed.

Intermediate portion 212 extends outward from handle 216 and is a length of catheter that can be moved through a patient. Outer sheath 218 and inner sheath 220 extend outward from handle 216 and form a portion of intermediate portion 212. Outer sheath 218 covers inner sheath 220.

Distal portion 214 extends from intermediate portion 212. Distal portion 214 includes bridge 222 and nosecone 224. Bridge 222 extends from inner sheath 220 towards nosecone 224. Nosecone 224 extends from bridge 222 to distal end 200B of delivery catheter 200. Bridge 222 is configured to hold shunt device 202. As shown in FIG. 7A, when delivery catheter 200 is in a sheathed state, outer sheath 218 will extend over and cover shunt device 202 on bridge 222. As shown in FIG. 7B, when delivery catheter 200 is in an unsheathed state, outer sheath 218 will be pulled back to expose bridge 222 and shunt device 202 on bridge 222. Nosecone 224 extends outward from bridge 222 and helps guide delivery catheter 200 through a patient's vasculature. Actuation rod 226, also called an actuation arm, extends through a lumen in inner sheath 220 and bridge 222. Actuation rod 226 emerges from side opening 228 in bridge 222 and connects to a first proximal arm of shunt device 202. Side opening 228 extends into a body of bridge 222. Notch 229 extends into the body of bridge 222 opposite side opening 228. Notch 229 is configured to seat a second proximal arm of shunt device 202. The second proximal arm can be retained on bridge 222 prior to deployment by a release wire (not shown) extending through a lumen of bridge 222 and through notch 229.

Delivery catheter 200 will be discussed below in more detail with respect to FIGS. 8A-9Q.

### Delivery Method 300 (FIGS. 8A-10C)

FIG. 8A is a flow chart showing steps for creating a puncture in tissue wall TW between coronary sinus CS and left atrium LA. FIG. 8B is a flow chart showing steps for implanting shunt device 202 in tissue wall TW between coronary sinus CS and left atrium LA. FIGS. 9A-9Q are schematic views showing the steps for implanting shunt device 202 in tissue wall TW between coronary sinus CS and left atrium LA. FIGS. 8A-9Q will be discussed together. FIGS. 8A-8B show method 300. FIG. 8A shows steps 302-316 of method 300. FIG. 8B shows steps 318-334 of method 300.

Step 302 includes advancing guidewire 230 into coronary sinus CS, as shown in FIG. 9A. Guidewire 230 can be inserted using traditional methods. Guidewire 230 is inserted into right atrium RA, through an ostium of coronary sinus CS, and then into coronary sinus CS. Optionally, a catheter having radiopaque markers can be inserted over guidewire 230 and imaging can be done to confirm placement of guidewire 230 in coronary sinus CS. Additionally, contrast can be injected into coronary sinus CS through the catheter to further confirm placement of guidewire 230 in coronary sinus CS. The catheter can then be removed once placement of guidewire 230 in coronary sinus CS is confirmed.

Step 304 includes advancing puncture catheter 232 over guidewire 230 to coronary sinus CS, as shown in FIG. 9B. Puncture catheter 232 is used to puncture tissue wall TW between coronary sinus CS and left atrium LA. Puncture catheter 232 includes catheter body 234 having opening 236 on a first side and balloon 238 on a second side opposite opening 236. Puncture catheter 232 can also include radiopaque markers 239 proximal and distal to opening 236 to confirm placement of puncture catheter 232 in coronary sinus CS. Puncture catheter 232 is advanced into coronary sinus CS so that opening 236 is facing tissue wall TW between coronary sinus CS and left atrium LA. Puncture catheter 232 shown in FIG. 9B is one example of a puncture catheter. In alternate examples, tissue wall TW can be punctured using other puncture catheters or other suitable mechanisms.

Step 306 includes inflating balloon 238 of puncture catheter 232, as shown in FIG. 9C. As balloon 238 is inflated, it will press against coronary sinus CS opposite of tissue wall TW. The inflation of balloon 238 will press puncture catheter 232 against tissue wall TW. Specifically, opening 236 will be pressed against tissue wall TW. Balloon 238 will anchor puncture catheter 232 in position in coronary sinus CS while a puncture is made in tissue wall TW. In alternate examples, any other suitable anchoring mechanism can be used instead of balloon 238. In further examples, step 306 is not needed.

Step 308 includes puncturing tissue wall TW between coronary sinus CS and left atrium LA, as shown in FIG. 9D. Puncture catheter 232 includes puncture arm 240 extending through a lumen in puncture catheter 232. Puncture arm 240 includes sheath 242 and needle 244 positioned in sheath 242 so that it extends out a distal end of puncture sheath 242. Puncture arm 240 can be advanced through puncture catheter 232 and out of opening 236 to puncture through tissue wall TW between coronary sinus CS and left atrium LA.

Puncture catheter 232 should be positioned in coronary sinus CS so that opening 236 of puncture catheter 232 is positioned 2-4 centimeters from the ostium of coronary sinus CS. This will position the puncture through tissue wall TW at the same location. The puncture, and ultimately the placement of shunt device 202 in the puncture, is positioned over the posterior leaflet of mitral valve MV.

Step 310 includes removing needle 244 from puncture catheter 232, as shown in FIG. 9E. Needle 244 can be removed by pulling it proximally through a lumen extending through needle sheath 242 of puncture arm 240. Needle 244 is fully removed from puncture catheter 232, leaving a lumen extending from a proximal end of puncture catheter 232 through a distal end of needle sheath 242.

Step 312 includes advancing guidewire 246 through puncture catheter 232 into left atrium LA, as shown in FIG. 9F. Specifically, guidewire 246 is advanced through a lumen extending through a proximal end of puncture catheter 232 and needle sheath 242 of puncture arm 240. Guidewire 246 is advanced into left atrium LA until it coils in left atrium LA, as shown in FIG. 9F. Once guidewire 246 is fully positioned in left atrium LA, puncture catheter 232 and guidewire 230 can be removed from left atrium LA and coronary sinus CS.

Step 314 includes advancing balloon catheter 248 over guidewire 246 and through the puncture in tissue wall TW, as shown in FIG. 9G. Balloon catheter 248 is advanced through the puncture in tissue wall TW so balloon 250 of balloon catheter 248 is positioned in the puncture in tissue wall TW. Balloon catheter 248 is shown as being a separate device from puncture catheter 232 in the example shown in FIG. 9G. However, in alternate examples, balloon catheter 248 can be inserted through puncture catheter 232 and through the puncture in tissue wall TW.

Step 316 includes inflating balloon 250 of balloon catheter 248 extending through the puncture in tissue wall TW, as shown in FIG. 9H. Balloon 250 extends along a distal portion of balloon catheter 248. As balloon 250 is inflated, it will expand and push open the tissue surrounding the puncture in tissue wall TW. The inflation of balloon 250 will cause the puncture in tissue wall TW to become a wider opening in which a shunt device can be positioned. Balloon 250 can then be deflated and balloon catheter 248 can be removed from left atrium LA and coronary sinus CS.

Step 318 includes advancing delivery catheter 200 over guidewire 246, as shown in FIG. 9I. Delivery catheter 200 has the general structure and design as discussed with reference to FIGS. 6-7B above. Delivery catheter 200 is inserted through coronary sinus CS, through the opening in tissue wall TW, and into left atrium LA. When delivery catheter 200 is properly positioned in tissue wall TW, nosecone 224 will be positioned in left atrium LA, and bridge 222 will extend through tissue wall TW between left atrium LA and coronary sinus CS. Nosecone 224 tapers from a smaller diameter at a distal end to a larger diameter at a proximal end. The taper of nosecone 224 helps to advance nosecone 224 through the opening in tissue wall TW and widens the opening as needed. Bridge 222 holds shunt device 202 (not shown in FIG. 9I) in a collapsed position on bridge 222. Bridge 222 is positioned in tissue wall TW so that shunt device 202 is generally positioned in the opening in tissue wall TW for deployment into the opening.

Step 320 includes withdrawing outer sheath 218 of delivery catheter 200 to release distal arms 252 of shunt device 202, as shown in FIG. 9J. Outer sheath 218 can be withdrawn to expose part of shunt device 202 held on bridge 222 of delivery catheter 200. As outer sheath 218 is withdrawn, distal arms 252 of shunt device 202 will be released and assume their preset shape. Delivery catheter 200 should be positioned in left atrium LA such that when outer sheath 218 is withdrawn to release distal arms 252 of shunts device 202, distal arms 252 of shunt device 202 are positioned in left atrium LA.

Step 322 includes pulling delivery catheter 200 proximally to seat distal arms 252 of shunt device 202 on tissue wall TW, as shown in FIG. 9K. Delivery catheter 200 can be gently pulled proximally to seat distal arms 252 of shunt device 202 on tissue wall TW in left atrium LA. A physician should stop gently pulling on delivery catheter 200 when resistance is sensed, indicating that distal arms 252 have come into contact with tissue wall TW. This will also position a central flow tube of shunt device 202 in the opening in tissue wall TW.

Step 324 includes withdrawing outer sheath 218 of delivery catheter 200 to expose proximal arms 254 of shunt device 202, as shown in FIG. 9L. Outer sheath 218 is withdrawn a set distance to fully expose shunt device 202, including proximal arms 254 of shunt device 202. Delivery catheter 200 should be positioned in left atrium LA, tissue wall TW, and coronary sinus CS so that proximal arms 254 will be positioned in coronary sinus CS when outer sheath 218 is withdrawn. Proximal arms 254 are constrained on bridge 222 of delivery catheter 200 and will not automatically assume their preset shape when outer sheath 218 is withdrawn.

Step 326 includes moving first proximal arm 254A of shunt device 202 towards tissue wall TW using actuation rod 226 of delivery catheter 200, as shown in FIG. 9M. Actuation rod 226 extends through a lumen in delivery catheter 200 and can be actuated forward to move first proximal arm 254A towards tissue wall TW.

Step 328 includes seating first proximal arm 254A on tissue wall TW, as shown in FIG. 9N. Actuation rod 226 of delivery catheter 200 is actuated fully outward to seat first proximal arm 254A on tissue wall TW. When first proximal arm 256A is seated on tissue wall TW, it will be positioned in coronary sinus CS.

Step 330 includes confirming placement of shunt device 202 in tissue wall TW. FIG. 9O illustrates a known method for confirming tissue confirmation, which includes injecting a contrast agent through a lumen extending through delivery catheter 200. The contrast agent can move through coronary sinus CS and left atrium LA. The contrast will highlight shunt device 202 under fluoroscopy to confirm proper placement of distal arms 252 and first proximal arm 254A of shunt device 202 on tissue wall TW. An alternative method of confirming tissue capture is discussed below.

Step 332 includes removing actuation rod 226 from first proximal arm 254A of shunt device 202, as shown in FIG. 9P. Actuation rod 226 can be held on and removed from first proximal arm 254A using any suitable mechanism. In the example shown in FIG. 9P, a release wire holds actuation rod 226 on first proximal arm 254A. The release wire can be withdrawn proximally to disconnect release wire from first proximal arm 254A. Actuation rod 226 can then be pulled proximally through a lumen of delivery catheter 200 to remove actuation rod 226 from coronary sinus CS.

Step 334 includes withdrawing delivery catheter 200 from coronary sinus CS and left atrium LA to release second proximal arm 254B of shunt device 202, as shown in FIG. 9Q. Second proximal arm 254B is held in place on bridge 222 in notch 229 formed in bridge 222. As delivery catheter 200 is withdrawn, second proximal arm 254B will be released from notch 229 in bridge 222 and take its preset shape. Specifically, second proximal arm 254B will seat upon tissue wall TW as it takes its preset shape. Second proximal arm 245B will be positioned in coronary sinus CS. After second proximal arm 254B is seated on tissue wall TW, shunt device 202 will be fully deployed in tissue wall TW, as shown in FIG. 10A (below). Delivery catheter 200 and guidewire 246 can then be removed from left atrium LA and coronary sinus CS.

Method 300 is one example of a method that can be used to implant shunt device 202 in tissue wall TW between left atrium LA and coronary sinus CS. Method 300 can include fewer, more, or different steps in alternate examples. Further, puncture catheter 232 and delivery catheter 200 are shown as being separate catheters in the example shown in FIGS. 9A-9Q but can be a single catheter in alternate examples.

Shunt devices must be anchored in place to avoid displacement during normal heart rhythms. Techniques are needed to confirmed proper placement of shunt devices during implantation.

FIG. 10A is a simplified perspective view of shunt device 202 properly seated between left atrium LA and coronary sinus CS. FIG. 10B is a simplified perspective view of shunt device 202 improperly seated between left atrium LA and coronary sinus CS. FIG. 10C is a simplified perspective view of shunt device 202 embolized in left atrium LA. FIGS. 10A-10C will be discussed together. FIGS. 10A-10C show shunt device 202, including distal arms 252 and proximal arms 254. FIGS. 10A-10C further show left atrium LA, coronary sinus CS, and tissue wall TW.

FIG. 10A shows shunt device 202 properly seated in tissue wall TW between left atrium LA and coronary sinus CS. As illustrated, distal arms 252 engage tissue wall TW and are positioned in left atrium LA and proximal arms 254 engage tissue wall TW and are positioned in coronary sinus CS. During deployment, one or more distal arms 252 or proximal arms 254 can be mis-seated. In one example, shunt device 202 could be improperly seated such that one or more of distal arms 252 is positioned in coronary sinus CS rather than in left atrium LA, as illustrated in FIG. 10B. For example, during an implantation procedure (e.g., during step 322 of method 300 as shown in FIG. 8B), a physician may pull delivery catheter 200 back too hard after distal arms 252 are released, causing all or a part of shunt device 202 to be pulled into coronary sinus CS. In another example, shunt device 202 could be improperly seated such that the entirety of shunt device 202 is located in left atrium LA, as illustrated in FIG. 10C. For example, during an implantation procedure (e.g., during step 322 of method 300 as shown in FIG. 8B), a physician may not pull delivery catheter 200 back far enough after distal arms 252 are released, so one or more of proximal arms 254 may be released in or pushed through to left atrium LA, causing shunt device 202 to embolize. Confirming tissue capture between the arms of a shunt device helps a physician to determine when it is safe to release the shunt device. As such, confirming proper seating of the shunt device during and/or following delivery also helps reduce the risk of embolization and/or need for redeployment. The present disclosure is directed to an apparatus for determining tissue capture of a shunt device. The disclosed apparatus can be used to confirm the presence of tissue wall TW between distal arms and proximal arms of the shunt device without infusing a contrast agent into the patient. As such, the disclosed apparatus is particularly important for procedures involving patients in which exposure to a contrast agent is contraindicated.

### Device and Method for Confirming Tissue Capture (FIGS. 11-16)

The occurrence of mis-seating of a shunt device can be decreased with accurate determination of tissue capture during delivery of the shunt device. In the present disclosure, a small alternating current (AC) signal can be used to measure electrical impedance between a reference electrode and an electrode positioned adjacent to at least one arm of the shunt device. Electrical impedance differs when measured through blood versus tissue. As such, measurement of the electrical impedance can be used to determine if tissue is captured between adjacent arms of the shunt device or if adjacent arms are seated on the same side of a tissue wall and separated by blood. In some examples, one or more existing wires of the previously disclosed delivery catheter that are connected to the shunt device, or that can be disposed adjacent to the shunt device, can function as the electrodes or can be used to supply the AC signal.

FIG. 11 is a simplified view of device 400 for determining tissue capture of shunt device 402 during deployment. Device 400, shunt device 402, distal arms 404A and 404B with terminal ends 406A and 406B, proximal arms 408A and 408B with terminal ends 410A and 410B, central flow tube 412, delivery catheter 414, bridge 416, first wire 418, second wire 420, actuation arm 422, distal tip 424, ground 426, AC signal 428, tissue walls TW, coronary sinus CS, and left atrium LA are shown in FIG. 11. A circuit can be formed between shunt device 402, first wire 418, second wire 420, distal tip 424, and tissue walls TW. A measurement of electrical impedance can be used to determine if the circuit is completed by tissue walls TW, as shown in FIG. 11, or by blood (shown in FIG. 12).

Shunt device 402 can have the structure and design of any suitable shunt device. Shunt device 402 can be substantially the same as or similar to shunt devices 100, 100′, and 202 illustrated in FIGS. 3A, 3B, 4, 5, 7B, 9J-9Q, and 10A-10C and described with respect thereto. Distal arms 404A and 404B are disposed on opposite sides of central flow tube 412. Proximal arms 408A and 408B are disposed on opposite sides of central flow tube 412. Distal arm 404A and proximal arm 408A are disposed on opposite axial ends of central flow tube 412 and configured to capture tissue walls TW of left atrium LA and coronary sinus CS therebetween. Distal arm 404B and proximal arm 408B are disposed on opposite axial ends of central flow tube 412 and configured to capture tissue walls TW of left atrium LA and coronary sinus CS therebetween (See, e.g., FIG. 10A). Shunt device 402 can be deployed according to method 300 with modification of step 330 relating to tissue capture confirmation as described further herein. Proximal arm 408A corresponds to first proximal arm 254A of shunt device 202 shown, for example, in FIG. 9M, moved toward tissue wall with actuation rod 226. Proximal arm 408B corresponds to second proximal arm 245B shown, for example, in FIG. 9Q, which is seated on tissue wall TW of coronary sinus CS as delivery catheter 200 is withdrawn.

Shunt device 402 is formed of an electrically conductive material. As previously described, shunt device 402 can be formed of a shape shape-memory material, such as nitinol (a nickel titanium alloy).

Device 400 includes delivery catheter 414. Delivery catheter 414 is one example of a delivery catheter that can be used to implant shunt device 402 into a patient. Delivery catheter 414 can be substantially similar to delivery catheter 200 shown in FIG. 7B and described with respect thereto. Delivery catheter 414 can take other forms in alternate examples. Bridge 416, first wire 418, second wire 420, and actuation arm 422 are shown. An outer sheath has been pulled back to release shunt device 402. As described with respect to delivery catheter 200 shown in FIG. 7B, an outer sheath holds shunt device 402 in a collapsed position on bridge 416 with distal arms 404A and 404B extending away from proximal arms 408A and 408B. As the outer sheath is pulled back, distal arms 404A and 404B return to their preset shape, curving toward proximal arms 408A and 408B, respectively, as shown in FIG. 11. Proximal arm 408B is secured to bridge 416. Proximal arm 408A is secured to actuation arm 422.

First wire 418 can be a stationary wire configured to temporarily retain proximal arm 408B of shunt device 402 on bridge 416. First wire 418 extends through a lumen (not shown) in delivery catheter 414. First wire 418 is formed of an electrically conductive material and is disposed in contact with proximal arm 408B to provide an electrical connection between first wire 418 and shunt device 402. Specifically, first wire 418 can be disposed in contact with terminal end 410B when terminal end 410B is seated on bridge 416. First wire 418 can remain in contact with proximal arm 408B until proximal arm 408B is released from delivery catheter 414. Proximal arm 408B can be released from delivery catheter 414 when delivery catheter is withdrawn from coronary sinus CS as shown in FIG. 9Q and described with respect thereto. First wire 418 can be formed, for example, from nitinol or other biocompatible conductive materials, including but not limited to platinum iridium, silver, gold, and stainless steel. As shown in FIG. 11, first wire 418 can be connected to ground 426. As described further herein, first wire 418 and shunt device 402 form a portion of the circuit used to determine tissue capture between distal arm 404A and proximal arm 408A.

Second wire 420 can be provided in actuation arm 422. Actuation arm 422 is one example of an actuation arm that can be used to position proximal arm 408A on tissue wall TW in coronary sinus CS. Actuation arm 422 can be substantially similar to actuation rod or arm 226 shown in FIGS. 7B, 9M, and 9N and described with respect thereto. Actuation arm 422 extends through a lumen (not shown) in delivery catheter 414 and can emerge from a side opening in bridge 416. Actuation arm 422 can be actuated forward to move proximal arm 408A toward tissue wall TW in coronary sinus CS. Proximal arm 408A of shunt device 402 can be connected to actuation arm 422 adjacent to distal tip 424 of actuation arm 422.

Second wire 420 can be a stationary wire formed of an electrically conductive material and configured to deliver the AC signal to the circuit. Second wire 420 can connect to AC signal 428 and extend through actuation arm 422 to distal tip 424. Distal tip 424 can also be formed of an electrically conductive material and can function as an electrode. Second wire 420 can be placed in contact with distal tip 424 to provide an electrical connection between second wire 420 and distal tip 424. Distal tip 424 is configured to engage tissue wall TW during deployment of proximal arm 408A in coronary sinus CS. The AC signal can be transmitted via distal tip 424 through tissue walls TW when distal tip 424 is placed in contact with tissue wall TW in coronary sinus CS.

When shunt device 402 is properly positioned between left atrium LA and coronary sinus CS, distal arm 404A of shunt device 402 is positioned on tissue wall TW of left atrium LA. Distal arm 404A and, specifically, terminal end 406A can function as the reference electrode or ground electrode. Actuation arm 422 can be actuated forward to position proximal arm 408A on tissue wall TW of coronary sinus CS. As actuation arm 422 is actuated forward, distal tip 424 can contact tissue wall TW of coronary sinus CS. As shown schematically in FIG. 11, a small AC current can be applied to second wire 420 and thereby distal tip 424. Tissue walls TW of coronary sinus CS and left atrium LA complete the circuit between distal tip 424 and distal arm 404A of shunt device 402. An electrical impedance sensor known in the art can be used to determine the electrical impedance based on voltage measured between distal tip 424 and terminal end 406A of shunt device 402. At a given current applied to the tissue (e.g., blood or tissue walls TW), a low voltage will correspond to a low impedance and a high voltage will correspond to a high impedance.

In some examples (shown in FIG. 15), second wire 420 can extend from actuation arm 422 to terminal end 410A of proximal arm 408A to locate the electrode on proximal arm 408A and thereby improve the accuracy of tissue capture determination. The electrode or terminal end of second wire 420 can be separated from terminal end 410A of proximal arm 408A by an insulating material (not shown) to avoid direct electrical connection between proximal arm 408A and second wire 420. For example, a biocompatible insulating film or pad (e.g., Kapton® polyamide) can be disposed between proximal arm 408A and second wire 420. The insulating material can be locally provided on proximal arm 408A or can extend from actuation arm 422 to proximal arm 408A with second wire 420. Second wire 420 can be configured to break or pull free from proximal arm 408A upon retraction of actuation arm 422.

The electrical impedance through tissue walls TW is substantially different from the electrical impedance through blood in coronary sinus CS and left atrium LA, which allows a physician to confirm when distal arm 404A of shunt device 402 and distal tip 424 of actuation arm 422 are each seated against tissue walls TW. Once tissue capture is confirmed, proximal arm 408A can be released as shown in FIG. 9P and described with respect thereto. Actuation arm 422 can then be withdrawn proximally through the lumen of delivery catheter 414 to remove actuation arm 422 from coronary sinus CS.

FIG. 12 a simplified view of device 400 during a process of determining tissue capture, wherein tissue capture is not confirmed. As previously described, during deployment, one or more distal arms 404A, 404B or proximal arms 408A, 408B can be mis-seated. In one example, shunt device 402 could be improperly seated such that one or more of distal arms 404A, 404B is positioned in coronary sinus CS rather than in left atrium LA, as illustrated in FIG. 12. For example, during an implantation procedure (e.g., during step 322 of method 300 as shown in FIG. 8B), a physician may pull delivery catheter 414 back too hard after distal arms 404A, 404B are released, causing all or a part of shunt device 402 to be pulled into coronary sinus CS. In another example, shunt device 402 could be improperly seated such that the entirety of shunt device 402 is located in left atrium LA, as illustrated in FIG. 10C. For example, during an implantation procedure (e.g., during step 322 of method 300 as shown in FIG. 8B), a physician may not pull delivery catheter 414 back far enough after distal arms 404A, 404B are released, such that one or more of proximal arms 408A, 408B may be released in or pushed through to left atrium LA, causing shunt device 202 to embolize. In either scenario, shunt device 402 fails to capture tissue walls TW between distal arm 404A and proximal arm 408A, and distal arm 404A of shunt device 402 can be separated from distal tip 424 of actuation arm 422 by the blood in coronary sinus CS or left atrium LA.

As shown schematically in FIG. 12, a small AC current can be delivered to distal tip 424. The blood in coronary sinus CS completes the circuit between distal tip 424 and distal arm 404A. The impedance sensor (shown in FIG. 13) can be used to determine the electrical impedance between distal tip 424 and terminal end 406A of shunt device 402 as previously described.

In some examples, the AC signal can be applied during deployment of actuation arm 422, allowing a physician to monitor the change in electrical impedance as distal tip 424 contacts tissue wall TW. In other examples, the AC signal can be applied after distal tip 424 has been fully deployed. A physician can differentiate the type of material separating electrodes of distal arm 404A of shunt device 402 and distal tip 424 of actuation arm 422 based on electrical impedance or a change in electrical impedance and can thereby determine whether shunt device 402 is properly seated. In some examples, an indicator light can be provided on a handle of delivery catheter 414 to inform the physician, based on a determined electrical impedance, whether shunt device 402 is properly seated.

FIG. 13 is a simplified schematic view of a handle of delivery catheter 414. FIG. 13 shows handle 430, first wire 418, second wire 420, programmable microchip 432, impedance sensor 433, and indicator lights 434. Handle 430 can be substantially similar to handle 216 shown in FIG. 6 and described with respect thereto. Impedance sensor 433 can be disposed in handle 430 and electrically connected to first wire 418 and second wire 420. Impedance sensor 433 is configured to determine an electrical impedance as known in the art. Programmable microchip 432 can be configured to receive and interpret a digital impedance signal. Programmable microchip 432 can be configured to analyze the electrical impedance signal using machine learning methods or with standard signal processing and analysis methods. This can include categorizing media (e.g., blood and tissue walls TW) using features including but not limited to amplitude, frequency, waveforms, capacitance/resistance/inductance, or real, and imaginary components derived from the impedance signal vector. Programmable microchip 432 can be electrically connected to one or more indicator lights 434. As previously discussed, electrical impedance across tissue walls TW is substantially different from electrical impedance across blood. In some examples, an electrical impedance range can be predetermined for each material (tissue walls TW and blood) and programmable microchip 432 can be configured to distinguish an electrical impedance across tissue walls TW from an electrical impedance across blood based on the predetermine ranges. In some examples, a baseline electrical impedance can be determined for a patient prior to full deployment of shunt device 402 when shunt device 402 is in blood and separated from tissue walls TW. Baseline measurements and calibration of the signal may be used to create a robust algorithm to provide accurate categorizations in all patients (e.g., patients with more fatty tissue, more muscular tissue, or thick or thin blood, etc.) Programmable microchip 432 can be configured to categorize the electrical impedance as indicating contact with blood or contact with tissue walls TW and indicate via one or more indicator lights 434 which material separates electrodes on distal arm 404A of shunt device 402 and distal tip 424 of actuation arm 422 or which material distal tip 424 is in contact with. For example, one or more different color indicator lights 434 can be provided on handle 430 to indicate when distal tip 424 contacts tissue walls TW. For example, an RGB LED can be configured to be lit red to indicate contact with blood and configured to be lit green to indicate contact with tissue walls TW. Indicator lights 434 can allow the physician to determine when shunt device 402 is properly seated and when shunt device 402 is mis-seated and requires adjustment or redeployment.

FIG. 14A is an enlarged view of portion 14A of FIG. 11. FIG. 13 shows first wire 418 in contact with proximal arm 408B of shunt device 402 on delivery catheter 414. In some examples, first wire 418 can extend overtop or through an opening in terminal end 410B of proximal arm 408B. First wire 418 can be a release wire configured to retain proximal arm 408A of shunt device 402 on delivery catheter 414. For example, first wire 418 can be threaded through one or more openings in proximal arm 408B (e.g., around a strut or through a hole at terminal end 410B). First wire 418 can be withdrawn from proximal arm 408A during deployment of shunt device 402, allowing proximal arm 408B to assume a predefined shape against tissue wall TW. In some examples, an outer sheath (not shown) on delivery catheter 414 can secure first wire 418 in a position of contact with proximal arm 408B.

FIG. 14B is an enlarged view of portion 14B of FIG. 11. Actuation arm 422, distal tip 424, proximal arm 408A, terminal end 410A, second wire 420, release wire 436, and connecting body 438 are shown. Actuation arm 422 includes distal tip 424 at a distal end. Connecting body 438 can be disposed proximal to distal tip 424. Second wire 420 can extend through actuation arm 422 to distal tip 424. Release wire 436 can extend through actuation arm 422 and connecting body 438. Release wire 436 can extend through an opening in terminal end 410A of shunt device 402 to retain proximal arm 408A on actuation arm 422. Release wire 436 can extend through a lumen of actuation arm 422 as previously described. As shown in FIG. 14B, connecting body 438 can include an opening to the lumen through which release wire 436 extends. Release wire 436 can be fed through an opening in terminal end 410A during assembly of shunt device 402 on delivery catheter 414 to capture proximal arm 408A. Release wire 436 can retain proximal arm 408A on actuation arm 422 until release wire 436 is withdrawn from actuation arm 422 following deployment of proximal arm 408A. Withdrawal of release wire 436 releases proximal arm 408A from actuation arm 422. Proximal arm 408A is free to assume a predefined shape against tissue wall TW.

Proximal arm 408A is electrically isolated from second wire 420 in actuation arm 422. In some examples, second wire 420 can be disposed in a separate lumen of actuation arm 422 and connecting body 438 can be formed of an electrically insulating material. In some examples, the portion of proximal arm 408A in contact with actuation arm 422 and release wire 436 can be coated with an electrically insulating material.

In some examples, first wire 418 can be connected to AC signal 428 and second wire 420 can be connected to ground 426. In some examples, second wire 420 can be eliminated and release wire 436 can serve as an electrode or can provide an electrical connection with distal tip 424 in actuation arm 422, which can serve as an electrode. For example, release wire 436 can be received in and connected to distal tip 424, which can be formed of an electrically conductive material. Alternatively, release wire 436 can extend adjacent to distal tip 424 and can be positioned to contact tissue wall TW upon deployment of actuation arm 422. Proximal arm 408A can be electrically isolated from release wire 436. For example, at least the portion of release wire 436 configured to contact terminal end 410A of proximal arm 408A can be sheathed in an electrically insulating material. In other examples, a biocompatible insulating film (e.g., Kapton® polyamide) can be provided on at least the portion of proximal arm 408A configured to contact release wire 436.

In another example, electrodes can be provided on any one or more locations of the shunt device, while remaining electrically isolated from the shunt device. In some examples, electrodes can be disposed to determine when pairs of arms of a shunt device contact a tissue wall TW. FIG. 15 is a simplified view of device 500 for determining tissue capture of shunt device 502 during deployment. FIG. 16 is an enlarged view of a portion of FIG. 15. Device 500, shunt device 502, distal arms 504A and 504B with terminal ends 506A and 506B, proximal arms 508A and 508B with terminal ends 510A and 510B, central flow tube 512, delivery catheter 514, bridge 516, first wire 518, second wire 520, third wire 522, fourth wire 524, first electrode 526, second electrode 528, third electrode 530, fourth electrode 532, insulating pad 534, actuation arm 536, distal tip 538, tissue walls TW, coronary sinus CS, and left atrium LA are shown in FIG. 15. Terminal end 506B, third electrode 530, insulating pad 534, and tissue wall TW are shown in FIG. 16.

First, second, third, and fourth wires 518, 520, 522, 524 have first, second, third, and fourth electrodes 526, 528, 530, 532, respectively. First, second, third, and fourth electrodes 526, 528, 530, 532 can be disposed on terminal ends 506A, 510A, 506B, 510B of shunt device 502, respectively. First, second, third, and fourth electrodes 526, 528, 530, 532 can be electrically isolated from shunt device 502 by insulating material disposed between first, second, third, and fourth electrodes 526, 528, 530, 532 and terminal ends 506A, 510A, 506B, 510B (e.g., insulating pad 534 on terminal end 506B).

As illustrated in FIG. 15, a circuit can be formed, for example, between electrodes 526 and 528 disposed on distal and proximal arms 504A and 508A, respectively, and tissue walls TW. A measurement of electrical impedance can be used to determine if the circuit is completed by tissue walls TW, as shown in FIG. 15, or by blood (e.g., similar to that shown in FIG. 12). Additional electrodes 530 and 532 disposed on distal and proximal arms 504B and 508B, respectively, can similarly be used to determine when distal and proximal arms 504B and 508B contact tissue walls TW. In some examples, third and fourth wires 522, 524 and electrodes 530, 532 can be omitted.

Shunt device 502 can have the structure and design of any suitable shunt device. Shunt device 502 can be substantially the same as or similar to shunt device 402 shown in FIG. 11 and described with respect thereto. In contrast to shunt device 402, shunt device 502 can include insulating film or pads (e.g., Kapton® polyamide) disposed on any of terminal ends 506A, 506B, 510A, 510B having electrodes 526, 528, 530, 532 to electrically isolate shunt device 502 from electrodes 526, 528, 530, 532 if shunt device 502 is formed of an electrically conductive material. Shunt device 502 can be deployed in substantially the same or similar manner as shunt device 402.

Device 500 can be substantially similar to device 400 shown in FIG. 11 and described with respect thereto. In contrast to device 400, delivery catheter 514 of device 500 can include two or more wires 518, 520, 522, 524 extending from a handle (e.g., similar to handle 430 shown in FIG. 13) to terminal ends 506A, 506B, 510A, 510B of shunt device 502.

As shown in FIG. 15, first, second, third, and fourth wires 518, 520, 522, 524 can extend through delivery catheter 514 and outward from openings in delivery catheter 514 to terminal ends 506A, 510A, 506B, 510B, respectively, of shunt device 502. Each of first, second, third, and fourth wires 518, 520, 522, 524 can be received in a lumen of delivery catheter 414. First, second, third, and fourth wires 518, 520, 522, 524 are electrically conductive. First, second, third, and fourth wires 518, 520, 522, 524 can be formed, for example, from nitinol or other biocompatible conductive materials, including but not limited to platinum iridium, silver, gold, and stainless steel. At least portions of first, second, third, and fourth wires 518, 520, 522, 524 disposed in contacting proximity to shunt device 502 can be sheathed in an electrically insulating material. Unsheathed terminal ends of first, second, third, and fourth wires 518, 520, 522, 524 can form electrodes 526, 528, 530, 532, respectively. Electrodes 526, 528, 530, 532 can be connected to distal arm 504A, proximal arm 508A, distal arm 504B, and proximal arm 508B, respectively, by a biocompatible insulating material (e.g., Kapton® polyimide film or pad) such that shunt device 502 is electrically isolated from electrodes 526, 528, 530, 532. An exposed side of electrodes 526, 528, 530, 532 opposite a connection to shunt device 502 is disposed to face tissue walls TW in deployment of shunt device 502. First, second, third, and fourth wires 518, 520, 522, 524 and/or the insulating material (insulating pad 534) can be configured to break free from shunt device 502 during withdrawal of delivery catheter 514 following deployment of shunt device 502.

In some examples, second wire 420 can be routed to proximal arm 508A of shunt device 502 via actuation arm 536 as previously described. In some examples, third wire 522 can be used to retain proximal arm 508B on delivery catheter 514 as previously described. Routing of first, second, third, and fourth wires 518, 520, 522, 524 through delivery catheter is not limited to the example shown.

A small AC current can be applied to any one of first, second, third, and fourth wires 518, 520, 522, 524. Electrodes disposed opposite the arm receiving the AC signal can be reference electrodes. For example, first and third wires 518 and 522 can be connected to ground and an AC signal can be provided to second and fourth wires 520 and 524. When shunt device 502 is properly positioned between left atrium LA and coronary sinus CS, distal arm 504A of shunt device 502 and electrode 526 is positioned on tissue wall TW in left atrium LA. Actuation arm 536 can be actuated forward to position proximal arm 508A and electrode 528 on tissue wall TW in coronary sinus CS. As shown schematically in FIG. 15, a small AC current can be applied to electrode 528 via second wire 520. Tissue walls TW between coronary sinus CS and left atrium LA complete the circuit between electrode 528 on proximal arm 508A and electrode 526 on distal arm 504A of shunt device 502. An impedance sensor can be used to determine if distal arm 504A and proximal arm 508A have captured tissue walls TW therebetween as previously described with respect to device 400. Similarly, tissue capture between proximal arm 504B and distal arm 508B can be confirmed by determining the electrical impedance between electrodes 530 and 532. The arrangement of electrodes is not limited to those shown. Other arrangements or combinations of electrodes on shunt device 502 and/or delivery catheter 514 are contemplated.

A programmable microchip can be used to categorize electrical impedance signals as previously described. In some examples a RGB LED on the handle of delivery catheter 514 can be used to indicate each electrode on shunt device 502 and the type of media in contact with each electrode. For example, an LED can be configured to be lit red to indicate contact with blood and can be configured to be lit green to indicate contact with tissue walls TW. Indicator lights can allow the physician to determine when shunt device 502 is properly seated and when shunt device 502 is mis-seated and requires adjustment or redeployment.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Any relative terms or terms of degree used herein, such as “substantially”, “essentially”, “generally”, “approximately” and the like, should be interpreted in accordance with and subject to any applicable definitions or limits expressly stated herein. In all instances, any relative terms or terms of degree used herein should be interpreted to broadly encompass any relevant disclosed embodiments as well as such ranges or variations as would be understood by a person of ordinary skill in the art in view of the entirety of the present disclosure, such as to encompass ordinary manufacturing tolerance variations, incidental alignment variations, transient alignment or shape variations induced by thermal, rotational or vibrational operational conditions, and the like. Moreover, any relative terms or terms of degree used herein should be interpreted to encompass a range that expressly includes the designated quality, characteristic, parameter or value, without variation, as if no qualifying relative term or term of degree were utilized in the given disclosure or recitation.

### Discussion of Detailed Embodiments

The following are non-exclusive descriptions of possible embodiments of the present invention.

A device for determining tissue capture of a shunt device includes a delivery catheter and a shunt device disposed on the delivery catheter. The shunt device is configured to be inserted into a puncture in a tissue wall and includes a central flow tube extending from a first axial end to a second axial end, a first distal arm attached to the first axial end of the central flow tube, and a first proximal arm attached to the second axial end of the central flow tube. The first distal arm and the first proximal arm configured to capture the tissue wall therebetween. The delivery catheter includes a first conductive wire and a second conductive wire. The first conductive wire is electrically connected to the shunt device and the second conductive wire is separated from the shunt device.

The device of the preceding paragraph can optionally include, additionally and/or alternatively, any one or more of the following features, configurations and/or additional components:

In a further embodiment of the preceding device, one of the first conductive wire and the second conductive wire can be connected to ground and the other of the first conductive wire and the second conductive wire can be connected to an alternating current signal.

In a further embodiment of any of the preceding devices, the shunt device is formed of an electrically conductive material and includes a second distal arm attached to the first axial end of the central flow tube and a second proximal arm attached to the second axial end of the central flow tube, wherein the second distal arm and the second proximal arm are configured to capture the tissue wall therebetween, and wherein the first conductive wire can be electrically connected to the second proximal arm.

In a further embodiment of any of the preceding devices, the delivery catheter can further include an actuation arm configured to position the first proximal arm, wherein the second conductive wire can be received in the actuation arm.

In a further embodiment of any of the preceding devices, the second conductive wire can be disposed adjacent to a terminal end of the first proximal arm.

In a further embodiment of any of the preceding devices, the actuation arm can include a conductive tip at a distal end and the second conductive wire can be electrically connected to the conductive tip.

In a further embodiment of any of the preceding devices, the first proximal arm can be retained on the actuation arm and an electrically insulating material can separate the first proximal arm from the second conductive wire.

In a further embodiment of any of the preceding devices, the first proximal arm can be retained on the actuation arm and an electrically insulating material can separate the second conductive wire from the first proximal arm.

In a further embodiment of any of the preceding devices, the first conductive wire can be connected to ground and the second conductive wire can be connected to the alternating current signal.

A further embodiment of any of the preceding devices can further include an electrical impedance sensor disposed in a handle of the delivery catheter, the electrical impedance sensor connected to the first conductive wire and the second conductive wire and configured to determine an electrical impedance between the second conductive wire and the first distal arm.

A further embodiment of any of the preceding devices can further include a programmable microchip in communication with the impedance sensor can be disposed in the handle and configured to categorize the electrical impedance as one of electrical impedance through blood and electrical impedance through a tissue wall.

A further embodiment of any of the preceding devices can further include an indicator light dispose on the handle of the delivery catheter, the indicator light in communication with the programmable microchip and configured to signal when tissue capture has occurred based on the categorization of the electrical impedance.

A method of determining tissue capture of a shunt device includes deploying a first distal arm of the shunt device into a puncture in a tissue wall, moving the first proximal arm toward the first distal arm to place the first proximal arm in a deployed position relative to the first distal arm, providing an alternating current signal to a first electrode, determining an electrical impedance between the first electrode and a second electrode, and determining if tissue has been captured between the first distal arm and the first proximal arm based on the electrical impedance. The shunt device includes central flow tube with the first distal arm disposed at a first axial end of the central flow tube and the first proximal arm disposed at a second axial end of the central flow tube. The first distal arm and the first proximal arm are configured to capture tissue walls therebetween. The first distal arm is free to engage the tissue wall when deployed. The deployed position of the first proximal arm is configured to capture the tissue wall the first distal arm and the first proximal arm if the tissue wall is present. One of the first electrode and the second electrode is disposed adjacent to the first proximal arm and the other of the first electrode and the second electrode is on the first distal arm.

The method of the preceding paragraph can optionally include, additionally and/or alternatively, any one or more of the following features, configurations, additional components, and/or steps:

In a further embodiment of the preceding method, the shunt device can be connected to a first conductive wire and the second electrode can be connected to a second conductive wire.

In a further embodiment of any of the preceding methods, one of the first conductive wire and the second conductive wire is connected to ground and the other of the first conductive wire and the second conductive wire is connected to the alternating current signal.

In a further embodiment of any of the preceding methods, the shunt device is formed of an electrically conductive material and further includes a second distal arm attached to the first axial end of the central flow tube and a second proximal arm attached to the second axial end of the central flow tube, with the second distal arm and the second proximal arm configured to capture the tissue wall therebetween, and wherein the first conductive wire can be electrically connected to the second proximal arm.

In a further embodiment of any of the preceding methods, the delivery catheter can further include an actuation arm configured to position the first proximal arm, wherein the second conductive wire can be received in the actuation arm.

In a further embodiment of any of the preceding methods, moving the first proximal arm to the deployed position can include moving the second electrode toward the first distal arm.

In a further embodiment of any of the preceding methods, the alternating current signal can be applied when the first proximal arm is in the deployed position.

In a further embodiment of any of the preceding methods, the second electrode can be disposed on a terminal end of the first proximal arm and separated from the terminal end by an insulating material.

In a further embodiment of any of the preceding methods, the second electrode can be a conductive tip disposed at a distal end of the actuation arm and wherein the second conductive wire is electrically connected to the conductive tip.

In a further embodiment of any of the preceding methods, the first proximal arm can be retained on the actuation arm and an electrically insulating material can separate the first proximal arm from the second conductive wire and the conductive tip.

In a further embodiment of any of the preceding methods, the first conductive wire can be connected to ground and the second conductive wire can be connected to the alternating current signal.

In a further embodiment of any of the preceding methods the electrical impedance can be determined by an electrical impedance sensor disposed in a handle of the delivery catheter and connected to the first conductive wire and the second conductive wire.

A further embodiment of any of the preceding methods can further include categorizing, via a programmable microchip disposed in the handle of the delivery catheter, the electrical impedance as one of electrical impedance through blood and electrical impedance through a tissue wall.

A further embodiment of any of the preceding methods can include signaling, via an indicator light disposed on the handle and connected to the programmable microchip, when tissue capture has occurred based on the electrical impedance.

The above method(s) can be performed on a living animal or on a simulation, such as on a cadaver, cadaver heart, anthropomorphic ghost, simulator (e.g., with body parts, heart, tissue, etc. being simulated).

A device for determining tissue capture of a shunt device configured to be inserted into a puncture in a tissue wall includes a delivery catheter and a shunt device disposed on the delivery catheter. The delivery catheter includes a first conductive wire having a first electrode and a second conductive wire having a second electrode. The shunt device includes a central flow tube extending from a first axial end to a second axial end, a first distal arm attached to the first axial end of the central flow tube, and a first proximal arm attached to the second axial end of the central flow tube, the first distal arm and the first proximal arm configured to capture the tissue wall therebetween. The first electrode is disposed on the first distal arm and separated from the shunt device by an insulating material. The second electrode is disposed adjacent to the first proximal arm and separated from the first proximal arm by one of spatial separation and an insulating material.

The device of the preceding paragraph can optionally include, additionally and/or alternatively, any one or more of the following features, configurations and/or additional components:

In an embodiment of the preceding device, one of the first conductive wire and the second conductive wire can be connected to ground and the other of the first conductive wire and the second conductive wire is connected to an alternating current signal.

In a further embodiment of any of the preceding devices, the delivery catheter can further include an actuation arm configured to position the first proximal arm. The second conductive wire can be received in the actuation arm.

In a further embodiment of any of the preceding devices, the actuation arm can include a conductive tip at a distal end and the second conductive wire can be electrically connected to the conductive tip.

In a further embodiment of any of the preceding devices, the first proximal arm can be retained on the actuation arm and an electrically insulating material can separate the first proximal arm from the second conductive wire.

In a further embodiment of any of the preceding devices, the second electrode can be disposed on the first proximal arm and separated from the first proximal arm by an insulating material.

In a further embodiment of any of the preceding devices, the shunt device further includes a second distal arm attached to the first axial end of the central flow tube and a second proximal arm attached to the second axial end of the central flow tube, the second distal arm and the second proximal arm configured to capture the tissue wall therebetween and the device can further include a third conductive wire having a third electrode. The third electrode can be disposed on one of the second distal arm and the second proximal arm and separated from the one of the second distal arm and the second proximal arm by an insulating material.

A further embodiment of any of the preceding devices can include a fourth conductive wire having a fourth electrode disposed on the other of the second distal arm and the second proximal arm. The fourth electrode can be separated from the other of the second distal arm and the second proximal arm by an insulating material.

A further embodiment of any of the preceding devices can include an electrical impedance sensor disposed in a handle of the delivery catheter and connected to the first conductive wire and the second conductive wire. The electrical impedance sensor configured to determine an electrical impedance between the first electrode and the second electrode.

A further embodiment of any of the preceding devices can include a programmable microchip in communication with the electrical impedance sensor and disposed in the handle of the delivery catheter. The programmable microchip can be configured to categorize the electrical impedance as one of electrical impedance through blood and electrical impedance through a tissue wall.

A further embodiment of any of the preceding devices can include an indicator light disposed on the handle of the delivery catheter, the indicator light in communication with the programmable microchip and configured to signal when tissue capture has occurred based on the calculated electrical impedance.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A system comprising:
a delivery catheter configured to be inserted into a puncture in a tissue wall, the delivery catheter comprising:
a first conductive wire; and
a second conductive wire; and
a shunt device disposed on the delivery catheter, the shunt device comprising:
a central flow tube extending from a first axial end to a second axial end;
a first distal arm attached to the first axial end of the central flow tube; and
a first proximal arm attached to the second axial end of the central flow tube, the first distal arm and the first proximal arm configured to capture the tissue wall therebetween;
wherein the first conductive wire is electrically connected to the shunt device; and
wherein the second conductive wire is separated from the shunt device.

2. The system of claim 1, wherein one of the first conductive wire and the second conductive wire is connected to ground and the other of the first conductive wire and the second conductive wire is connected to an alternating current signal.

3. The system of claim 2, wherein the shunt device further comprises:
a second distal arm attached to the first axial end of the central flow tube; and
a second proximal arm attached to the second axial end of the central flow tube, the second distal arm and the second proximal arm configured to capture the tissue wall therebetween;
wherein the first conductive wire is electrically connected to the second proximal arm; and
wherein the shunt device is formed of an electrically conductive material.

4. The system of claim 2, wherein the delivery catheter further comprises an actuation arm configured to position the first proximal arm, wherein the second conductive wire is received in the actuation arm.

5. The system of claim 4, wherein the actuation arm comprises a conductive tip at a distal end, and wherein the second conductive wire is electrically connected to the conductive tip.

6. The system of claim 4, wherein the first proximal arm is retained on the actuation arm and the second conductive wire is disposed adjacent to a terminal end of the proximal arm, wherein an electrically insulating material separates the first proximal arm from the second conductive wire.

7. The system of claim 4 and further comprising:
an electrical impedance sensor disposed in a handle of the delivery catheter, the electrical impedance sensor connected to the first conductive wire and the second conductive wire and configured to determine an electrical impedance between the second conductive wire and the first distal arm; and
a programmable microchip in communication with the electrical impedance sensor and disposed in the handle of the delivery catheter, the programmable microchip configured to categorize the electrical impedance as one of electrical impedance through blood and electrical impedance through the tissue wall.

8. The system of claim 7 and further comprising an indicator light disposed on the handle of the delivery catheter, the indicator light in communication with the programmable microchip and configured to signal when tissue capture has occurred based on the categorization of the electrical impedance.

9. A delivery catheter for an implantable device, the delivery catheter comprising:
a first conductive wire electrically connected to the implantable device or extending to a first electrode disposed adjacent to a first portion of the implantable device;
a second conductive wire extending to a second electrode disposed adjacent to a second portion of the implantable device; and
an electrical impedance sensor connected to the first conductive wire and the second conductive wire and configured to determine an electrical impedance between the first portion and the second portion of the implantable device to detect tissue capture between the first portion and the second portion of the implantable device.

10. The delivery catheter of claim 9, wherein the second electrode is connected to the second portion of the implantable device by an insulating material.

11. The delivery catheter of claim 10, wherein the first electrode is connected to the first portion of the implantable device by an insulating material.

12. The delivery catheter of claim 9 and further comprising:
an actuation arm having a distal tip, the actuation arm extendable from the delivery catheter to position the second portion of the implantable device on the tissue wall;
wherein the distal tip is formed of an electrically conductive material and wherein the second conductive wire is disposed in contact with the distal tip.

13. The delivery catheter of claim 12, wherein the first conductive wire is electrically connected to the implantable device and the second conductive wire is electrically isolated from the implantable device.

14. The delivery catheter of claim 9 and further comprising:
a programmable microchip in communication with the electrical impedance sensor, the programmable microchip configured to detect tissue capture between the first portion and the second portion of the implantable device based on the determined electrical impedance; and
a handle at a proximal end, the handle comprising the electrical impedance sensor and the programmable microchip, the programmable microchip configured to categorize the electrical impedance as one of electrical impedance through blood and electrical impedance through the tissue wall.

15. The delivery catheter of claim 14 and further comprising an indicator light disposed on the handle, the indicator light in communication with the programmable microchip and configured to signal when tissue capture has occurred based on the categorization of the electrical impedance.

16. A system comprising:
a delivery catheter configured to be inserted into a puncture in a tissue wall, the delivery catheter comprising:
a first conductive wire having a first electrode; and
a second conductive wire having a second electrode; and
a shunt device disposed on the delivery catheter, the shunt device comprising:
a central flow tube extending from a first axial end to a second axial end;
a first distal arm attached to the first axial end of the central flow tube; and
a first proximal arm attached to the second axial end of the central flow tube, the first distal arm and the first proximal arm configured to capture the tissue wall therebetween;
wherein the first electrode is disposed on the first distal arm, the first electrode separated from the shunt device by an insulating material; and
wherein the second electrode is disposed adjacent to the first proximal arm, the second electrode separated from the first proximal arm by a spatial separation and/or an insulating material.

17. The system of claim 16, wherein the delivery catheter further comprises an actuation arm configured to position the first proximal arm, wherein the second conductive wire is received in the actuation arm and is electrically insulated from the first proximal arm, and wherein the second conductive wire is electrically connected to a conductive tip at a distal end of the actuation arm or disposed on the first proximal arm and separated from the first proximal arm by an insulating material.

18. The system of claim 17, and further comprising:
a second distal arm attached to the first axial end of the central flow tube;
a second proximal arm attached to the second axial end of the central flow tube, the second distal arm and the second proximal arm configured to capture the tissue wall therebetween; and
a third conductive wire having a third electrode;
wherein the third electrode is disposed on one of the second distal arm and the second proximal arm, the third electrode separated from the one of the second distal arm and the second proximal arm by an insulating material.

19. The system of claim 18, and further comprising:
a fourth conductive wire having a fourth electrode;
wherein the fourth electrode is disposed on the other of the second distal arm and the second proximal arm, the fourth electrode separated from the other of the second distal arm and the second proximal arm by an insulating material.

20. The system of claim 16, and further comprising:
an electrical impedance sensor disposed in a handle of the delivery catheter, the electrical impedance sensor connected to the first conductive wire and the second conductive wire and configured to determine an electrical impedance between the first electrode and the second electrode;
a programmable microchip in communication with the electrical impedance sensor and disposed in the handle of the delivery catheter, the programmable microchip configured to categorize the electrical impedance as one of electrical impedance through blood and electrical impedance through the tissue wall; and
an indicator light disposed on the handle of the delivery catheter, the indicator light in communication with the programmable microchip and configured to signal when tissue capture has occurred based on the categorization of the electrical impedance.
